(19) United States
(12) Patent Application Publication
(10) Pub. No.: US 2006/0280748 A1
(43) Pub. Date: Dec. 14, 2006

(54) Plasma or serum fraction for treatment or prevention of abnormal cell proliferation

(76) Inventor: Robert W. Buckheit JR., Myersville, MD (US)

Correspondence Address: KING & SPALDING LLP, 1180 PEACHTREE STREET, ATLANTA, GA, 30309, US

(21) Appl. No.: 11/410,537

(22) Filed: Apr. 24, 2006

Related U.S. Application Data
(60) Provisional application No. 60/674,381, filed on Apr. 22, 2005.

(51) Int. Cl. A61K 39/42; A61K 39/395; A61K 39/21; A61K 39/00

(52) U.S. Cl. 424/155.1; 424/184.1; 424/208.1; 424/160.1

(57) ABSTRACT

The present invention relates to a plasma or serum fraction derived from a mammal exposed to an inoculant, which fraction has been depleted of one or more high molecular weight proteins present in the unprocessed plasma or serum, as well as to a method to treat and/or prevent abnormal cell proliferation, such as cancer, with the plasma or serum fraction.

### CROSS REFERENCE TO RELATED APPLICATIONS

This application claims priority to U.S. Provisional Patent Application No. 60/674,381 filed on Apr. 22, 2005.

### FIELD OF THE INVENTION

The present invention provides compositions, methods and uses for treating and preventing diseases or disorders characterized by abnormal cell proliferation, including cancer. The invention also includes methods of producing the compositions of the present invention.

### BACKGROUND OF THE INVENTION

In multicellular organisms, homeostasis is supported by a carefully regulated balance between cell proliferation and cell death (apoptosis). Cell proliferation is a highly regulated process involving positive regulation by growth factors and proto-oncogenes as well as negative regulation by tumor suppressor genes (Rozengurt E. “Growth factors and cell proliferation” Curr. Opin. Cell Biol. 1992 4:161; Levine A J. “The tumor suppressor genes” Annu. Rev. Biochem. 1993 62:623). A number of diverse human pathologies are known to be associated with uncontrolled cell proliferation, including cancer and other non-cancerous disorders.

Cancer

Cancer is a diverse class of diseases or disorders characterized by uncontrolled division of cells and the ability of these cells to invade other tissues, either by direct growth into adjacent tissue through invasion or by implantation into distant sites by metastasis. In general, cancers are divided into four types based on their origin: carcinomas, lymphomas, leukemias and sarcomas. Overall, the most common cancers include bladder, breast, colon, rectal, endometrial, leukemia, lung cancer, melanoma, non-Hodgkin's lymphoma, pancreatic, prostate, skin and thyroid cancer.

Carcinomas are tumors (i.e., neoplasms) arising from epithelial tissue, such as glands, breast, skin, and linings of the urogenital, digestive, and respiratory systems. Breast cancer is a particularly common form of carcinoma that originates in the ducts or lobules of the breast. Invasive cancer cells that spread (i.e., by moving through blood or lymphatic vessels) beyond the breast area to other parts of the body (i.e., bone or lung), are known as metastatic breast cancer. Prostate cancer is the most common male malignancy in developed countries and the second leading cause of cancer mortality (Rebillard X, Tretarre B, Villers A. “The epidemiology of prostate cancer” Rev Prat. 2003 53(20):2224-8; Greenlee R T et al. “Cancer Statistics, 2001” Cancer J. Clin. 2001 51:15-38). While prostate cancer is typically not a fatal disease, it can cause a variety of uncomfortable symptoms that severely impact quality of life.

Lymphomas are cancers that originate in the lymph nodes and spleen that are characterized by the excessive production of lymphocytes. Lymphomas are generally grouped to include Non-Hodgkin's lymphoma and Hodgkin's disease, with the former much more prevalent than the latter. Hodgkin's disease is defined histopathologically by the presence of the malignant Reed Sternberg cells in the cancerous area. Non-Hodgkin's lymphoma refers to a group of nearly thirty lymphomas classified by lymphatic cell type and growth rate (Harris N L, Jaffe E S, Kiebold J, Flandrin G, Muller-Hermelink H K, Vardiman J. “Lymphoma classification-from controversy to consensus: the REAL and WHO Classification of lymphoid neoplasms” Ann Oncol. 2000; 11(suppl 1):S3-S10).

Leukemias originate in the bone marrow and are generally classified as lymphocytic or myeloid, depending on the type of leukocyte involved. Leukemias are further classified as acute (i.e., a rapidly progressing disease that involves immature leukocytes) or chronic (i.e, a slower proliferation involving mature white cells). Myeloma is highly related to leukemia as a cancer of plasma cells; a type of white blood cell found throughout the body but primarily in the bone marrow.

Sarcomas are the least common form of cancer (2%). Some originate in bone while others originate in the soft tissues including muscles, tendons, fibrous tissues, fat, blood vessels nerves, and synovial tissues (tissues around joints). Clinically relevant malignant sarcomas include osteosarcoma (cancerous tumor of the bone), neurofibrosarcomas (malignant tumors of nerve sheath origin), liposarcomas (malignant lesions of adipose tissue), rhabdomyosarcomas (malignant tumors derived from striated muscle cells), among others.

Cancer is a disease with profound personal and economic costs. A huge number of people are either directly or indirectly affected by the disease. The National Cancer Institute estimates that approximately 1,372,910 new cases of cancer were diagnosed in 2005. See American Cancer Society: Cancer Facts and Figures 2005 (available on-line at www.cancer.org). In the same year, approximately 570,280 Americans were expected to die of cancer, more than 1,500 people per day. Cancer remains the second leading cause of death in the United States, producing 1 of every 4 deaths. The economic costs of cancer are also great. The National Institutes of Health estimate overall costs for cancer in 2004 at $189.8 billion, including $64.9 billion for direct medical costs (i.e., health expenditures); $16.9 billion for indirect morbidity costs (i.e., cost of lost productivity due to illness); and $103.5 billion for indirect mortality costs (i.e., cost of lost productivity due to premature death.

Despite the investment of billions of dollars, a (consistent) cure remains illusive. Cancer treatments generally include chemotherapy, radiation therapy, immunotherapy and gene therapy. Each is associated with particular advantages and disadvantages. Chemotherapy involves the use of anti-cancer drugs to treat cancerous cells. In general, chemotherapeutic agents can be divided into three main categories based on their mechanism of action. The first class of chemotherapeutic agents work by directly damaging the DNA in the cell nucleus. Examples include cisplatin, daunorubicin, doxorubicin, and etoposide. The second class of chemotherapy agents hinder nucleotide synthesis. Examples include methotrexate, mercaptopurine, fluorouracil, and hydroxyurea. The third class of chemotherapeutic agents affects the synthesis or breakdown of mitotic spindles. Examples include vinblastine, vincristine, and pacitaxel. The use of chemotherapeutic agents to treat cancer suffers several major limitations. Lack of specific cytocidal action presents one such limitation. Chemotherapy reaches both normal and cancerous cells within the body. In general, efficacy is greatest against actively cycling cells like cancer cells. The unintended impact on normal cells, however, produces unpleasant side effects. Common side effects of chemotherapeutic agents include nausea and vomiting, hair loss, anemia, reduced blood clotting, mouth sores, and increased likelihood of developing infections. The development of drug resistance is also a significant problem. Cancers that initially respond to chemotherapeutic drugs or to antihormonal therapies frequently relapse in a resistant form.

Radiation therapy involves the use of ionizing radiation that damages genetic material at the target site, thereby preventing further cellular division. Like chemotherapy, radiation inflicts damage on healthy normal tissues. Burning of the skin and hair loss are common side effects of radiation therapy. More problematically, radiotherapy can cause secondary cancers after the primary cancer has been treated. Other secondary diseases such as pneumonitis and radiation fibrosis may also occur. Radiation therapy is also associated with both acute and delayed disturbances in nutritional status.

Immunotherapy involves the stimulation or enhancement of the immune response to fight cancer. Cytokines that are used to treat cancer include interferons and interleukins. Interferon-alpha is now approved by the FDA and is commonly to treat cancers including multiple myeloma, chronic myelogenous leukemia, hairy cell leukemia, and malignant melanoma. Interferon-beta and interferon-gamma are also under investigation. Interleukins are used to treat cancer include IL-2 for renal cancer and melanoma. Tumor necrosis factor has also been shown to have anti-tumor activity. Use of cytokines for the treatment of cancer, however, is associated with unpleasant side effects, which include malaise and flu-like syndromes, which are magnified at high doses.

Monoclonal antibodies are also used to treat cancer. Rituximab (Rituxan) has been used in the treatment of non-Hodgkin's lymphoma, while trastuzumab (Herceptin, Genetech) is used against certain breast cancers. Bone and marrow stem cell transplants have also been used to treat certain types of cancer. Stem cell transplantation from marrow is a standard therapy for selected patients with leukemia, lymphoma and myeloma. Autologus transplants (transplants of the patient's own bone marrow) can be used to treat chronic myeloid leukemia, certain acute leukemias, lymphoma and myeloma as well as certain solid tumors including breast cancer and testicular cancer.

Cancer vaccines continue to be evaluated in the treatment of cancer. Therapeutic vaccination involves the use of a cancer antigen in combination with other immune enhancing substances to further stimulate the patient's immune response against the disease. The antigen can come from the patient's own cancer cell or from cancer cells grown in the laboratory. Despite decades of experimental work however, results from cancer vaccine studies are mixed. Challenges facing the development of an effective cancer vaccine include identification of better antigens as well as strategies for package the antigen to enhance the immune response.

Gene therapy is involves modifying the genetic material of living cells to fight disease. Gene therapy was originally viewed as an approach for treating hereditary disease, but has become the focus of efforts to treat acquired diseases such as cancer. Despite considerable research and investment, however, no gene therapy for cancer has show sufficient has yet been approved (Gottesman M M. “Cancer gene therapy: an awkward adolescence” Cancer Gene Ther. 2003 10(7):501-8). Successful clinical implementation likely awaits vectors capable of specific and efficient gene delivery to target cells (Douglas J T. “Cancer gene therapy” Technol Cancer Res Treat. 2003 2(1):51-64).

Even where treatments are available, cost of treatment remains high. See Brown M L, Riley G F, Schussler N, Etzioni R D. Estimating health care costs related to cancer treatment from SEER-Medicare data. Med Care 2002 August; 40(8 Suppl):IV-104-17. Lack of health insurance and other barriers prevent many Americans from receiving optimal care.

Other Diseases of Abnormal Cell Proliferation

Benign tumors are characterized by abnormal cell proliferation but are not malignant, recurrent, invasive or progressive. Yet, due to metabolic effects or critical location (e.g., the brain), certain benign tumors can have devastating consequences.

Abnormal proliferation of smooth muscle cells is involved in atherosclerosis and restenosis. (Sanz-Gonzalez S M, Poch E, Perez-Roger I, Diez-Juan A, Ivorra C, Andres V. “Control of vascular smooth muscle cell growth by cyclin-dependent kinase inhibitory proteins and its implication in cardiovascular disease” Front Biosci. 2000 1:5:D619-28; Andres V. “Control of vascular smooth muscle cell growth and its implication in atherosclerosis and restenosis (review)” Int J Mol Med. 1998 2(1):81-9). Vascular smooth muscle cells within the medial layer of elastic arteries proliferate in response to physiological and pathological stimuli causing neointimal thickening during spontaneous atherosclerosis and vessel renarrowing (restenosis) after angioplasty. Abnormal cell proliferation is also involved in lymphangiomyomatosis (LAM); a progressive lung disease characterized by overgrowth of smooth muscle inside the lung. Most patients die within 15 years of symptom onset (Reid J K, Rees H, Cockcroft D. “Long term survival in a patient with pulmonary lymphangioleiomyomatosis” Can Respir J. 2002 9(5):342-6).

Rheumatoid arthritis (RA) is a chronic inflammatory disease characterized by hyperplasia of the synovial lining cells which cartilage destruction in the RA joint (Harris E D J. “Rheumatoid arthritis. Pathophysiology and implications for therapy” N Engl J Med 1990 322:1277-89; Volin M V and Koch A E. “Cell cycle implications in the pathogenesis of rheumatoid arthritis” Frontiers in Bioscience 2000 5:D594-601). The altered rates of proliferation and apoptosis of RA synovial cells result in the hyperplasia of synovial tissue and in concert with the chronic inflammatory environment ultimately lead to the destruction of the RA joint.

Mastocytosis encompasses a range of disorders characterized by over-proliferation and accumulation of tissue mast cells. It is a disease of complex etiology. The skin is frequently directly involved in mastocytosis (cutaneous mastocytosis or CM) but the skeleton, gastrointestinal tract, bone marrow, and central nervous system may also be involved. Aggressive mastocytosis is a form of systemic mast cell disease characterized by organ infiltration, bone lesions. eosinophilia and lymphadenopathies.

Mesangial cell proliferation is a prominent feature of many human glomerular diseases including IgA nephropathy, membranoproliferative glomerulonephritis (GN), lupus nephritis and diabetic nephropathy (Jefferson J, Johnson R. “Experimental mesangial proliferative glomerulonephritis (the anti-Thy-1.1 model)” J Nephrol 1999 12: 297-307).

Psoriasis is a relatively common chronic (and non-infectious) skin disease in which epidermal regeneration has become unregulated. It is generally believed that psoriasis is caused by impairment in the immune system, enzymes, and other biochemical substances that regulate skin cell division. One or more genetic abnormalities maybe involved. There are a variety of types of psoriasis, with the most common known as plaque psoriasis.

Various other abnormal cell proliferation disease and disorders are well known in the art.

Passive Immunity

The principle of passive immunity provides that neutralizing antibodies from a different organism may be used to prevent or treat disease in another organism of the same or different species. Passive immunization has a long history in the treatment of disease. The earliest form of passive immunity involved the use of serum therapy in the late 1800's. Behring and Kitasato were the first to use passive immunization in the treatment of diphtheria in 1890. In the 1920's and 30's, sera produced in animals (e.g., horse, rabbit, sheep) was used to treat patients with measles and scarlet fever. At that time, serum was not known to contain antibodies; rather it was only observed that serum produced a beneficial therapeutic effect.

Blood is now understood as a liquid tissue containing cells, proteins, salts and various amounts of organic substances. The term plasma refers to that portion of the blood that remains after the cellular elements (i.e., red blood cells, white blood cells) have been removed, typically by centrifugation. Serum, in contrast, refers to the portion of the blood that remains after both the cellular elements and the clotting proteins are removed. Clotting will occur naturally when blood is added to a test tube, but can also be stimulated by clotting activators such as calcium. Serum contains water (98%), protein (6-8%), salts (0.8%), lipids (0.6%) and glucose (0.1%). Other molecules, including metabolites, hormones and enzymes are also present. Serum should be therefore be distinguished from plasma, although the two terms are sometimes used interchangeably incorrectly. Those skilled in the art of blood collection are familiar with the differences between the two, which determine how blood samples are collected. To prevent conversion to serum, plasma is typically collected in tubes coated with an anticoagulant, or anticoagulant is added to the blood shortly after collection. Common additives used to prevent coagulation include heparin, EDTA, citrate or oxalate. Serum is collected in uncoated tubes and permitted to clot, or in tubes with clotting activators to speed the process.

Serum contains a complex mixture of proteins that contains various proteins ranging in concentration over at least 9 orders of magnitude (Adkins, J N et al., Mol Cell Proteomics 2002 1(12):947-55). The most abundant serum proteins are albumins, which account for between 60-80% of total serum protein. Albumin is a small protein produced by the liver and responsible for transport of small molecules, such as calcium, around the body. Albumin also helps keep blood fluids from leaking out into tissue. Globulins are a second form of protein found in large quantities in serum. Larger than albumin, the globulins can be classified according to three major types: alpha, beta and gamma. Alpha and beta globulins mainly carry various lipids, lipid-soluble hormones and vitamins, and other lipid-like substances in the plasma. The alpha-1 fraction includes alpha-1 anti-trypsin and thyroxine binding globulin. The alpha-2 fraction contains haptoglobin, ceruloplasmin, HDL, and alpha-2 macroglobulin. The beta fraction includes transferrin. The gamma globulins consist primarily of the immunoglobulins (i.e., IgA, IgM, IgG). Protein levels may vary somewhat based on, for example, disease or nutritional state.

The early use of serum therapy declined in popularity with the discovery of antibodies, and the development of strategies such as pooling and monoclonal antibody technology. Numerous extraction and purification strategies have been developed to isolate serum proteins with various degrees of specificity. These strategies exploit the differences among proteins with respect to such characteristics as size, charge, and binding affinity, among others. Representative technologies include chromatography (i.e., gel filtration, affinity and ion exchange), precipitation and gel electrophoresis. Robert K. Scopes, Protein Purification: Principles and Practice, 3rd edition, Springer Verlag (1994). These techniques can be used alone or in combination. For example, ionic precipitation (i.e., using ammonium sulfate) is most often used early in a purification to permit some subset of proteins to be fractionated from the whole based on common solubility parameters, and is often followed by chromatography. Ionic precipitation can be used to obtain a crude extract of proteins that can then be further purified.

WO 03/004049 (Dalgleish) describes the use of a anti-HLA and/or anti-FAS antibody enriched fraction of goat serum for the treat or prevent diseases involving a proliferative immune response or inappropriately high levels of HLA. Representative diseases include HIV and tropical cancers such as lung, pancreas, liver, bowel, lymph nodes, and skin cancer. The antibody-enriched serum extract is prepared by inoculating a goat with lysed HIV virus (heat killed), every week for four weeks. See also WO 03/064472 (Dalgleish).

Given the serious personal and economic burden associated causes by disorders of abnormal cell proliferation, there exists a need for new therapeutic strategies.

Therefore, it is an object of the present invention to provide a composition for the treatment or prevention of disorders of abnormal cell proliferation, such as cancer.

It is another object of the present invention to provide a method and use for treating and preventing disorders of abnormal cell proliferation, as cancer.

It is a further object of the present invention to provide a composition, method and use for treating and preventing disorders of abnormal cell proliferation that consists of a simple, economical regime of therapy that encourages compliance and presents minimal side effects.

It is another object of the present invention to provide a method for the manufacture of a composition or medicament for the treatment and prevention of disorders of abnormal cell proliferation.

### SUMMARY OF THE INVENTION

A composition, method and use for the treatment and prevention of disorders of abnormal cell proliferation, including cancer, are disclosed. The composition, method and use provide a plasma or serum fraction derived from a mammal exposed to an inoculant, which fraction has been depleted of one or more high molecular weight proteins or biological agents present in the unprocessed plasma or serum. Without being bound by any particular theory, it is believed that one or more biological agents present in the plasma or serum fraction depleted of one or more high molecular weight proteins or biological agents will generate a beneficial plethoric effect in vivo against disorders of abnormal cell proliferation, such as cancer.

The composition of the present invention, in one embodiment an immunoglobulin depleted fraction of plasma or serum derived from a mammal exposed to an inoculant (e.g., a cancer-bearing inoculant such as a breast cancer antigen-bearing inoculant), differs from the prior art reviewed above which utilizes immunoglobulin concentrates or purified antibodies for the treatment of cancer. The composition, method and use of the present invention provide a simple, cost-effective regime for treatment of disorders of abnormal cell proliferation, either alone or in combination with conventional treatments with activity against diseases of abnormal cell proliferation.

Accordingly, one aspect of the invention is a composition useful in the treatment or prevention of a disorder of abnormal cell proliferation, which is a plasma or serum fraction derived from a mammal exposed to an inoculant, which fraction has been depleted of one or more high molecular weight proteins or biological agents present in the unprocessed plasma or serum.

The inoculant used to generate the plasma or serum fraction of the present invention may vary. In one embodiment, the inoculant is an abnormal cell proliferation-bearing inoculant. Representative, non-limiting abnormal cell proliferation-bearing inoculants include cancer-bearing inoculants, atherosclerosis-bearing inoculants, restenosis-bearing inoculants, rheumatoid arthritis-bearing inoculants, and psoriasis-bearing inoculants.

In a particular embodiment, the abnormal cell proliferation-bearing inoculant is a cancer-bearing inoculant. Cancer-bearing inoculants include, without limitation, the blood, plasma or serum of an individual with cancer, a cancer cell, a cancer cell lysate, or proteins or other components (e.g., carbohydrates) of a cancer cell.

In another particular embodiment, the plasma or serum fraction is derived from a mammal exposed to a cancer antigen-bearing inoculant (e.g., a breast-cancer antigen).

In another embodiment, the inoculant is a viral-bearing inoculate such as an HIV-bearing inoculant.

The high molecular weight protein(s) or biological agent(s) depleted from the plasma or serum fraction of the present invention may vary. Representative, non-limiting high molecular weight proteins include immunoglobulin, albumin, transferrin, haptoglobin and lipoproteins.

The term “depletion” is used to indicate a reduction in the amount of a compound(s) or molecule(s) (e.g., high molecular weight proteins) in a given sample after the sample is treated according to the method of the present invention.

In one embodiment, the plasma or serum fraction is depleted of approximately 1-10%, 10-20%, 20-30%, 30-40%, 40-50%, 50-60%, 60-70%, 70-80%, 80-90%, or 90-100% of the one or more high molecular weight proteins present in the unprocessed plasma or serum sample.

In a particular embodiment, the present invention is a composition useful in the treatment of prevention of a disorder of abnormal cell proliferation derived from a mammal exposed to an inoculant, which fraction has been depleted of immunoglobulin present in the unprocessed plasma or serum.

In a more particular embodiment, the plasma or serum fraction is depleted of approximately 1-10%, 10-20%, 20-30%, 30-40%, 40-50%, 50-60%, 60-70%, 70-80%, 80-90%, or 90-100% of the immunoglobulin present in the unprocessed plasma or serum sample.

Another aspect of the invention is a composition useful in the treatment or prevention of a disorder of abnormal cell proliferation, which is a plasma or serum fraction derived from a mammal exposed to an inoculant, which fraction has been depleted of two or more different high molecular weight proteins present in the unprocessed plasma or serum.

In a particular embodiment, the plasma or serum fraction has been depleted of immunoglobulin and albumin.

In a more particular embodiment, the plasma or serum fraction is depleted of approximately 1-10%, 10-20%, 20-30%, 30-40%, 40-50%, 50-60%, 60-70%, 70-80%, 80-90%, or 90-100% of the immunoglobulin and albumin present in the unprocessed plasma or serum sample.

In another aspect, the invention is a composition useful in the treatment or prevention of a disorder abnormal cell proliferation, which is a plasma or serum fraction derived from a mammal exposed to an inoculant, which fraction has been depleted of proteins or biological agents with a molecular weight greater than about 30 kD present in the unprocessed plasma or serum.

In yet another aspect, the invention is a composition useful in the treatment or prevention of a disorder of abnormal cell proliferation, which is a plasma or serum fraction derived from a mammal exposed to an inoculant, which fraction has been depleted of proteins or biological agents with a molecular weight greater than about 50 kD present in the unprocessed plasma or serum.

In a still further aspect, the invention is a composition useful in the treatment or prevention of a disorder abnormal cell proliferation, which is a plasma or serum fraction derived from a mammal exposed to an inoculant, which fraction has been depleted of proteins or biological agents with a molecular weight greater than approximately 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 kD present in the unprocessed plasma or serum.

Another aspect of the present invention is a method of treating or preventing a disorder of abnormal cell proliferation in a subject such as a human by administering a therapeutic amount of a plasma or serum fraction derived from a mammal exposed to an inoculant, which fraction has been depleted of one or more high molecular weight proteins or biological agents present in the unprocessed plasma or serum, either alone or in combination or alternation with another anti-abnormal cell proliferation agent or agent that treats a related condition.

In a particular embodiment, the subject is a human.

The composition of the present invention can be administered by any effective means, including but not limited to, subcutaneous, parenteral, intravenous, intraarterial or oral administration. In a particular embodiment, the composition is administered by subcutaneous injection.

In one embodiment, the invention is a method of treating or preventing cancer in a subject such as a human by administering a therapeutic amount of a plasma or serum fraction derived from a mammal exposed to an inoculant (e.g., a cancer-bearing inoculant or an HIV-bearing inoculant), which fraction has been depleted of one or more high molecular weight proteins, either alone or in combination or alternation with another anti-cancer agent or agent that treats a related condition.

In a particular embodiment, the invention is a method of treating or preventing cancer (e.g., breast cancer or prostate cancer) in a subject such as a human by administering a therapeutic amount of a plasma or serum-fraction derived from a mammal exposed to an inoculant (e.g., a cancer-bearing inoculant or HIV-bearing inoculant), which fraction has been depleted of immunoglobulin, either alone or in combination or alternation with another anti-cancer agent or agent that treats a related condition.

In another particular embodiment, the invention is a method of treating or preventing cancer (e.g., breast or prostate cancer) in a subject such as a human by administering a therapeutic amount of a plasma or serum-fraction derived from a mammal exposed to an inoculant (e.g., a cancer-bearing inoculant or an HIV-bearing inoculant), which fraction has been depleted of immunoglobulin and albumin, either alone or in combination or alternation with another anti-cancer agent or agent that treats a related condition.

Another aspect of the present invention is a method of preparing a plasma or serum fraction useful in the treatment or prevention of a disorder of abnormal cell proliferation, involving (a) exposing a mammal to an inoculant; (b) allowing time for the mammal to respond to the inoculant and to produce one or more beneficial biologic agents in the blood; and (c) obtaining the plasma or serum; (d) processing the plasma or serum to isolate the anti-abnormal cell proliferation activity from one or more high molecular weight proteins present in the unprocessed plasma or serum.

In one embodiment, the mammal is an ungulate. In a preferred embodiment, the mammal is a goat.

In a further embodiment, the mammal is not susceptible to infection with the inoculant.

The process used to isolate the anti-abnormal cell proliferation activity from the one or more high molecular weight proteins present in the unprocessed plasma or serum may vary. The process may include, without limitation, fractionation methods such as fractional precipitation, dialysis and ultrafiltration, and/or chromatographic fractionation. In a particular embodiment, the process includes ammonium sulfate precipitation. In another embodiment, the process includes gel filtration chromatography, ion exchange chromatography or affinity chromatography. The process may involve a single fractionation step or multiple fractionation steps involving the same or different fractionation methods.

In one embodiment, the process involves sequential ammonium sulfate precipitation in combination with chromatographic fractionation.

In a particular embodiment, the plasma or serum fraction is processed to isolate the anti-abnormal cell proliferation activity from proteins or biological agents with a molecular weight greater than approximately 50 kD.

In another particular embodiment, the plasma or serum fraction is processed to isolate the anti-abnormal cell proliferation activity from proteins or biological agents with a molecular weight greater than approximately 30 kD.

### BRIEF DESCRIPTION OF THE FIGURES

FIG. 1 is a graphical representation of the protein profile obtained as described in Example 13 from a DG-10 desalting column of Week 3 serum from an animal inoculated as described in Example 11. The column was equilibrated with buffer A, 3 ml of serum was loaded onto the column and 1 ml fractions were collected. The protein concentration of each fraction was determined and the results were plotted vs. the approximate elution volume. Fractions representing milliliters 4 through 9 were pooled for DEA-blue chromatography.

FIG. 2 shows Coomassie blue stain of partially purified protein serum fractions. Protein from serum and partially purified fractions (as described in Example 13) was subjected to electrophoresis on a 6 to 18% polyacrylamide Tris-SDS gel. Following electrophoresis, the gel was stained with Coomassie G-250 to visualize the proteins. BR and P are Bio Rad broad range pre-stained molecular weight markers, and Pierce TriChromRanger molecular weight markers, respectively. The molecular weight in kilodaltons of the BioRad markers are indicated on the left hand side of the figure. IgG is 2 μg of purified goat IgG obtained from the NIH AIDS Research and Reference Reagent Program.

FIG. 3 is an immunoblot of partially purified serum fractions with anti-goat IgG, as described in Example 13. Protein from serum and partially purified fractions was subjected to electrophoresis on a 6 to 18% polyacrylamide Tris-SDS gel. Following electrophoresis, the gel was stained with Coomassie G-250 to visualize the proteins. BR and P are Bio Rad broad range pre-stained molecular weight markers, and Pierce TriChromRanger molecular weight markers, respectively. The molecular weight in kilodaltons of the BioRad markers are indicated on the left hand side of the figure. IgG is 2 μg of purified goat IgG obtained from the NIH AIDS Research and Reference Reagent Program.

FIG. 4 is a graphical representation of the chromatographic profile for the DEAE-Blue column fractionation of the 66% ammonium sulfate pellet detailed in Example 15, from an animal inoculated as described in Example 11. The blue trace monitors absorbance at 254 nm vs time and represents the protein elution profile. The red trace monitors eluate conductivity vs. time and represents the ionic concentration of the wash or elution buffer.

FIG. 5 shows SDS-PAGE from the partial fractionation detailed in Example 15, from an animal inoculated as described in Example 11. Five micrograms of total protein for each fraction was electrophoresed on an 8-16% polyacrylamide gel. The gel was stained with Bio Safe Commassie stain over night at room temperature and destained with water. The image was captured using an Alpha Inotech gel imager. The molecular weights of dye-labeled protein standards (BioRad) is indicated on the left of the figure.

### DETAILED DESCRIPTION OF THE INVENTION

The present invention includes compositions, methods and uses for treating and preventing disorders of abnormal cell proliferation and related conditions. In a particular embodiment, the compositions and methods of the present invention are useful for treating and preventing cancer, including breast and prostate cancer. In addition, these compositions and methods can be used to treat a variety of disorders associated with abnormal cell proliferation that are not cancerous, including atherosclerosis, restenosis and other disorders detailed below.

As used herein, “treatment” is an approach for obtaining beneficial or desired results, including clinical results. For purposes of this invention, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The following are non-limiting embodiments of how to carry out the invention. Given the description of the invention provided throughout this text, one of ordinary skill can modify the steps below without deviating form the spirit or scope of the invention.

A. Process for Producing the Plasma or Serum Fraction

(i) Selection of Animal

The plasma or serum fraction may be prepared using a mammal that produces the effective product according the process described in detail herein. In a particular embodiment, the mammal produces an effective product post-inoculation.

In one embodiment, the mammal or animal is not susceptible to the infection or disease caused by the infectious agent or disease agent used to produce inoculant (as detailed further below). For example, when HIV is used as an inoculant (as described further below), the animal should not be susceptible to infection with HIV.

Non-limiting examples of animals suitable for use in producing the plasma or serum fraction of the present invention include cow, rabbit, dog, mouse, goat, lamb, sheep, horse, deer, pig, mouse, chicken and the like (e.g., Bora goats). In a particular embodiment, the mammal is an ungulate or hoofed-mammal. Non-limiting examples of ungulates include goat, sheep, horses and cows.

In a particularly preferred embodiment, the mammal is a goat.

(ii) Inoculation of the Animals

Any inoculant suitable for generating the plasma or serum fraction can be used in the present invention. The inoculant may contain a single immunogen or multiple immunogens, of the same r different. The inoculant may be, for example, an abnormal cell proliferation-bearing inoculant or a viral-bearing inoculant, or may include immunogens of abnormal cell proliferation in combination with viral immunogens.

In a particular embodiment, the inoculant is a viral-bearing inoculant. Non-limiting examples of viral-bearing inoculants include the blood, plasma or serum of an individual infected with a virus, a viral lysate, purified virus or naturally occurring or synthetic viral proteins or peptides (glycosylated or unglycosylated). The virus may be one of the following:

In one embodiment of the present invention, the viral inoculant is a Herpesviridae bearing inoculant, a Retroviridae bearing inoculant, a Flaviviridae bearing inoculant, an Orthomyxoviridae bearing inoculant, a Paramyxoviridae bearing inoculant, a Togaviridae bearing inoculant, a Picornaviridae bearing inoculant, a Coronaviridae bearing inoculant, an Adenoviridae bearing inoculant, a Poxyiridae bearing inoculant, a Hepadnaviridae bearing inoculant, a Reoviridae bearing inoculant, a Parvoviridae (including a Parvovirinae and/or Densovirinae bearing inoculant), a Rhabdoviridae bearing inoculant, a Bunyaviridae bearing inoculant, a Bromoviridae bearing inoculant, a Totiviridae bearing inoculant, a Tectiviridae bearing inoculant, a Plasmaviridae bearing inoculant, a Myoviridae bearing inoculant, a Siphoviridae bearing inoculant, a Podoviridae bearing inoculant, a Rudiviridae bearing inoculant, a bearing inoculant, a Corticoviridae bearing inoculant, a Lipothrixviridae bearing inoculant, a Plasmaviridae bearing inoculant, a Fuselloviridae bearing inoculant, a Phycodnaviridae bearing inoculant, an Iridoviridae bearing inoculant, a Polydnaviridae bearing inoculant, a Polyomaviridae bearing inoculant, a Papillomaviridae bearing inoculant, a bearing inoculant, an Ascoviridae bearing inoculant, a Baculoviridae bearing inoculant, a Nimaviridae bearing inoculant, an Asfarviridae bearing inoculant, an Inoviridae bearing inoculant, a Microviridae bearing inoculant, a Geminiviridae bearing inoculant, a Circoviridae bearing inoculant, a Nanoviridae bearing inoculant, a Pseudoviridae bearing inoculant, a Metaviridae bearing inoculant, a Caulimoviridae bearing inoculant, a Cystoviridae bearing inoculant, a Birnaviridae bearing inoculant, a Totiviridae bearing inoculant, a Chrysoviridae bearing inoculant, a Partitiviridae bearing inoculant, a Hypoviridae bearing inoculant, a Filoviridae bearing inoculant, a Bornaviridae bearing inoculant, an Arenaviridae bearing inoculant, a Leviviridae bearing inoculant, a Dicistroviridae bearing inoculant, a Sequiviridae bearing inoculant, a Comoviridae bearing inoculant, a Potyviridae bearing inoculant, a Caliciviridae bearing inoculant, an Astroviridae bearing inoculant, a Nodaviridae bearing inoculant, a Tetraviridae bearing inoculant, a Tombusviridae bearing inoculant, an Arteriviridae bearing inoculant, a Roniviridae bearing inoculant, a Bromoviridae bearing inoculant, a Closteroviridae bearing inoculant, a Bamaviridae bearing inoculant, a Luteoviridae bearing inoculant, a Narnaviridae bearing inoculant, a Pospiviroidae bearing inoculant, an Avsunviroidae bearing inoculant, and/or a prion bearing inoculant.

In a sub-embodiment of the present invention, the viral inoculant is a Herpesviridae bearing inoculant. In a particular embodiment of the invention, the viral inoculant is an HSV-1 or HSV-2 bearing inoculant. In another particular embodiment of the present invention, the viral inoculant is a human herpesvirus 3 (varicella-zoster virus) bearing inoculant. In yet another particular embodiment of the present invention, the viral inoculant is a CMV-bearing inoculant. In a still another particular embodiment of the present invention, the viral inoculant is an EBV-bearing inoculant. In a still another particular embodiment of the present invention, the viral inoculant is a human herpesvirus 6-bearing inoculant. In a still another particular embodiment of the present invention, the viral inoculant is a human herpesvirus 7-bearing inoculant. In a still another particular embodiment of the present invention, the viral inoculant is a human herpesvirus 8 (Kaposi's sarcoma-associated herpesvirus)-bearing inoculant.

In a sub-embodiment of the present invention, the viral inoculant is a Retroviridae bearing inoculant. In a particular embodiment of the invention, the viral inoculant is an HIV-bearing inoculant, wherein the HIV includes the many clades, types and subtypes of HIV. In a particular embodiment of the invention, the viral inoculant is an HIV-1 bearing inoculant (Clade A, B, C, D, F, H, and/or 0) and/or HIV-2 (Clade A and/or B) bearing inoculant. In another particular embodiment of the invention, the viral inoculant is a Human T-lymphotropic virus 2 (HTLV-2) bearing inoculant.

In another sub-embodiment of the present invention, the viral inoculant is a Flaviviridae bearing inoculant. In a particular embodiment of the invention, the viral inoculant is a flavivirus bearing inoculant, wherein the flavivirus is, for example, a Dengue virus (Dengue virus, Dengue virus type 1, Dengue virus type 2, Dengue virus type 3, Dengue virus type 4), a Japanese encephalitis virus (Alfuy Virus, Japanese encephalitis virus, Kookaburra virus, Koutango virus, Kunjin virus, Murray Valley encephalitis virus, St. Louis encephalitis virus, Stratford virus, Usutu virus, West Nile Virus), a Modoc virus, a R10 Bravo virus (Apoi virus, R10 Brovo virus, Saboya virus), a Ntaya virus, a Tick-Borne encephalitis (tick born encephalitis virus), a Tyuleniy virus, an Uganda S virus and/or a Yellow Fever virus. In another particular embodiment of the invention, the viral inoculant is a hepacivirus bearing inoculant, wherein the hepacivirus is, for example, a hepatitis C virus (HCV) and/or its many clades, types and subtypes. In yet another particular embodiment of the invention, the viral inoculant is a pestivirus bearing inoculant, wherein the pestivirus is, for example, Bovine Viral Diarrhea Virus-2 (BVDV-2), Pestivirus type 1 (including BVDV), Pestivirus type 2 (including Hog Cholera Virus) and/or Pestivirus type 3 (including Border Disease Virus).

In yet another sub-embodiment of the present invention, the viral inoculant is an Orthomyxoviridae bearing inoculant. In a particular embodiment of the invention, the viral inoculant is an Influenzavirus A bearing inoculant. In another particular embodiment of the invention, the viral inoculant is an Influenzavirus B bearing inoculant. In yet another particular embodiment of the invention, the viral inoculant is an Influenzavirus C bearing inoculant. In yet another particular embodiment of the invention, the viral inoculant is an Influenzavirus D bearing inoculant.

In yet another sub-embodiment of the present invention, the viral inoculant is a Paramyxoviridae bearing inoculant. In a particular embodiment of the invention, the viral inoculant is a Paramyxovimae bearing inoculant. In an even more particular embodiment of the invention, the viral inoculant is a paramyxovirus, wherein the paramyxovirus is, for example, a Sendai virus, such as human parainfluenza virus 1 and human parainfluenza virus 3. In an even more particular embodiment of the invention, the viral inoculant is a human parainfluenza virus 1 bearing inoculant. In another even more particular embodiment of the invention, the viral inoculant is a human parainfluenza virus 3 bearing inoculant. In another particular embodiment of the invention, the viral inoculant is a rubulavirus bearing inoculant. In an even more particular embodiment of the invention, the viral inoculant is a human parainfluenza virus 2 bearing inoculant. In another even more particular embodiment of the invention, the viral inoculant is a human parainfluenza virus 4 bearing inoculant. In an even more particular embodiment of the invention, the viral inoculant is a mumps virus bearing inoculant. In another particular embodiment of the invention, the viral inoculant is a morbillivurs bearing inoculant. In more particular embodiment of the invention, the viral inoculant is a measles virus bearing inoculant. In another particular embodiment of the invention, the viral inoculant is a Pneumovirnae bearing inoculant. In a more particular embodiment of the invention, the viral inoculant is a respiratory syncytial virus (RSV) bearing inoculant.

In yet another sub-embodiment of the present invention, the viral inoculant is a Coronaviridae bearing inoculant. In a particular embodiment of the invention, the viral inoculant is a human respiratory coronavirus (HCV-229E) bearing inoculant. In another particular embodiment of the invention, the viral inoculant is a human respiratory coronavirus (HCV-OC43) bearing inoculant. In yet another particular embodiment of the invention, the viral inoculant is a torovirus bearing inoculant, such as a human torovirus bearing inoculant.

In yet another sub-embodiment of the present invention, the viral inoculant is a Togaviridae bearing inoculant. In a particular embodiment of the invention, the viral inoculant is an alphavirus bearing inoculant. In another particular embodiment of the invention, the viral inoculant is a rubivirus bearing inoculant. In an even more particular embodiment of the invention, the viral inoculant is a Rubella virus bearing inoculant. In another even more particular embodiment of the invention, the viral inoculant is a Sindbis virus bearing inoculant. In another even more particular embodiment of the invention, the viral inoculant is Eastern/Western encephalitis virus bearing inoculant.

In yet another sub-embodiment of the present invention, the viral inoculant is a Picornaviridae bearing inoculant. In a particular embodiment of the invention, the viral inoculant is a human rhinovirus bearing inoculant, wherein the human rhinoviruses can be any one of the at least 105 serotypes (a classification scheme based on the variation of surface epitopes), which represent the most common etiological agent for the common cold. In a particular embodiment of the invention, the viral inoculant is an enterovirus. In an even more particular embodiment of the invention, the viral inoculant is a Human polioviruses 1, 2, and 3 (A23-echovirus; echo=Enteric Cytopathic Human Orphan viruses) (3 serotypes), Human coxsackieviruses A1-22, 24 (23 serotypes), Human coxsackieviruses B1-6 (swine vesicular disease virus is very similar to coxsackie B5 virus) (6 serotypes), Human echoviruses 1-7, 9, 11-27, 29-34 (30 serotypes; these viruses show a seasonal, epidemic pattern of infection primarily associated with meningitis, paralysis (usually less severe than acute poliomyelitis), and myocarditis), Human enteroviruses 68-71 (4 serotypes), and/or Vilyuisk virus (1 serotype) bearing inoculant. In a particular embodiment of the invention, the viral inoculant is cardiovirus bearing inoculant, wherein the cardiovirus can be, for example, an encephalomyocarditis (EMC) virus (a mouse virus that can infect humans, elephants, and squirrels; includes mengovirus, Maus-Elberfield virus, and the Columbia virus) and Theiler's murine encephalocyelitis (TME) virus (TO, GDVII). In another particular embodiment of the invention, the viral inoculant is hepatovirus bearing inoculant. In yet another particular embodiment of the invention, the viral inoculant is human hepatitis virus A bearing inoculant. In yet another particular embodiment of the invention, the viral inoculant is a severe acute respiratory syndrome (SARS) Co—V bearing inoculant.

In yet another sub-embodiment of the present invention, the viral inoculant is a Hepadnaviridae bearing inoculant. In a particular embodiment of the invention, the viral inoculant is a human hepatitis B virus (HBV) bearing inoculant.

In yet another sub-embodiment of the present invention, the viral inoculant is an Adenoviridae bearing inoculant. In a particular embodiment of the invention, the viral inoculant is a human adenovirus A, B, C, D, E, and/or F bearing inoculant.

In yet another sub-embodiment of the present invention, the viral inoculant is an Arenaviridae bearing inoculant. In a particular embodiment of the invention, the viral inoculant is a human hepatitis D virus (HDV) bearing inoculant.

In yet another sub-embodiment of the present invention, the viral inoculant is a Caliciviridae bearing inoculant. In a particular embodiment of the invention, the viral inoculant is a human hepatitis E virus bearing inoculant.

In another embodiment of the invention, the inoculant is an abnormal cell proliferation-bearing inoculant. In one, the inoculant is a cancer-bearing inoculant. Non-limiting examples of cancer-bearing inoculants include the blood, plasma or serum of an individual with cancer, a cancer cell, a cancer cell lysate, or proteins or other components (e.g., carbohydrates) of a cancer cell.

A number of alterations occur in the cell during tumorigenesis (e.g., enzymes, receptors, membrane antigens, etc.). In general, certain molecules characteristic of cancer cells are either unique or more abundant than those characteristic of normal or non-cancerous cells. Some of these may be secreted while others are membrane-associated molecules. Tumor-specific antigens (TSA, also called tumor-specific transplantation antigens, TSTA, or tumor rejection antigens, TRA) are present on the surface of cancer cells, but not on non-tumor cells. Tumor-associated antigens (TAA) are more common. TAA are found on tumor cells and on normal cells during fetal life (onco-fetal antigens), after birth in selected organs, or in many cells but at much lower concentration than on tumor cells.

Analysis of spontaneous immune responses against cancer in humans has led to the identification of a large number of tumor antigens (Boon T, Old L J. Curr Opin Immunol (1997) 9:681-683). Collectively, molecular and cellular techniques investigators have defined more than 500 tumor antigens. The majority of these antigens are classified into categories on the basis of their expression pattern, function, or origin: (i) cancer-testis (CT) antigens, e.g., MAGE (1, 2 & 3) and NY-ESO-1, which are aberrantly expressed in tumor cells but that, with the exception of germ cells, are silent in normal cells; (ii) differentiation antigens of the melanocyte lineage, e.g., Melan A/MART-1, tyrosinase, and gp100; (iii) mutational antigens, e.g., MUM-1, p53, and CDK4; (iii) overexpressed “self” antigens, e.g., HER2/neu and p53; (iv) and viral antigens, e.g., HPV and EBV.

In a particular embodiment, the present invention is a composition useful for the treatment or prevention of cancer derived from a mammal exposed to a cancer antigen-bearing inoculant, which fraction has been depleted of one or more high molecular weight proteins or biological agents.

Commonly recognized cancer antigens include, without limitation, gp100, carcinoembryonic antigen (CEA), HER-2, mucins (i.e., MUC-1), prostate-specific antigen (PSA), and prostatic acid phosphate, squamous cell carcinoma antigen 1(SCCA-1), (proteinT4-A), squamous cell carcinoma antigen 2 (SCCA-2), ovarian carcinoma antigen CA125 (1A1-3B) (KIAA0049), mucin 1 (tumor associated mucin), (carcinoma-associated mucin), (polymorphic epithelial mucin), (PEM), (PEMT), (episialin), (tumor associated epithelial membrane antigen), (EMA), (H23AG), (peanut reactive urinary mucin), (PUM), (breast carcinoma associated antigen DF3), CTCL tumor antigen sel-1, CTCL tumor antigen se14-3. CTCL tumor antigen se20-4, CTCL tumor antigen se20-9, CTCL tumor antigen se33-1, CTCL tumor antigen se37-2, CTCL tumor antigen se57-1, CTCL tumor antigen se89-1, Prostate-specific membrane antigen, 5T4 oncofetal trophoblast glycoprotein, MAGE-C1 (cancer/testis antigen CT7), MAGE B-1 antigen (MAGE-XP ANTIGEN) (DAM10), MAGE-B2 antigen (DAM6), MAGE-2 antigen, MAGE-4a antigen, MAGE-4b antigen, Colon cancer antigen NY-CO-45, Lung cancer antigen NY-LU-12 variant A, Cancer associated surface antigen, Adenocarcinoma antigen ART1, Paraneoplastic associated brain-testis-cancer antigen (onconeuronal antigen MA2; paraneoplastic neuronal antigen), Neuro-oncological ventral antigen 2 (NOVA2), Hepatocellular carcinoma antigen gene 520, tumor associated antigen CO-029, Tumor-associated antigen MAGE-X2, Synovial sarcoma, X breakpoint 2, Squamous cell carcinoma antigen recognized by T cell, Serologically defined colon cancer antigen 1, Serologically defined breast cancer antigen NY-BR-15, Serologically defined breast cancer antigen NY-BR-16, Chromogranin A; parathyroid secretory protein 1, DUPAN-2, CA 19-9, and CA 72-4, and CA 195, among others.

Certain antigens are known to be characteristic of certain types of cancer. Certain representative, non-limiting examples of these antigens are shown below:

| Antigen | Antigen Function | Expression |
|---|---|---|
| Cyclin- | Cell cycle regulator | Melanoma |
| dependent | | |
| kinase 4 | | |
| b-catenin | Signal transduction | Melanoma |
| Caspase-8 | Apoptosis regulator | Squamous cell |
| | | carcinoma |
| MAGE-1 | Normal testicular | Melanoma, breast, |
| MAGE-3 | proteins | glioma tumors; |
| Tyrosinase | Melanin synthesis | Melanoma |
| Surface Ig | BCR | Lymphoma |
| idiotype | | |
| Her-2/neu | Receptor tyrosine | Breast and |
| | kinase | ovarian cancer |
| MUC-1 | Underglycosylated | Breast and |
| | mucin | pancreatic tumors |
| HPV E6 and E7 | Viral gene products | Cervical carcinoma |

In a particular embodiment, the inoculant is a breast cancer antigen-bearing inoculant.

In another particular embodiment, the inoculant is a prostate cancer antigen-bearing inoculant.

Other cancer antigens are thought to be more universal, that is, present in all major forms of cancer. Teleomerase is one such universal cancer antigen. Homo sapiens telomerase reverse transcriptase (hTRT) is a tumor-associated antigen expressed in the vast majority of human tumors. NY-ESO-1 is a cancer/testis (CT) antigen that has been found in a wide variety of cancers, including melanoma, breast cancer, lung cancer, synovial sarcoma, and hepatocellular carcinoma.

In a particular embodiment, the cancer-associated immunogen is autologous.

In another embodiment, the abnormal cell proliferation-bearing inoculant is a atherosclerosis-bearing inoculant. Representative, non-limiting examples of atherosclerosis bearing inoculants include the blood, plasma or serum of a patient suffering from atherosclerosis.

In a further embodiment, the abnormal cell proliferation-bearing inoculant is a restenosis-bearing inoculant. Representative, non-limiting examples of restenosis-bearing inoculants include the blood, plasma or serum of a patient suffering from restenosis.

In a further embodiment, the abnormal cell proliferation-bearing inoculant is a rheumatoid arthritis-bearing inoculant. Representative, non-limiting examples of rheumatoid arthristis-bearing inoculants include the blood, plasma or serum of a patient suffering from rheumatoid arthristis.

In a still further embodiment, the abnormal cell proliferation-bearing inoculant is a psoriasis-bearing inoculant. Representative, non-limiting examples of psoriasis-bearing inoculants include the blood, plasma or serum of a patient suffering from psoriasis.

In another embodiment of the present invention, the inoculant is a prion bearing inoculant. In a particular embodiment of the invention, the inoculant is a prion bearing inoculant, wherein the prion is the causative agent of a spongiform encephalopathy such as Scrapie, Bovine spongiform encephalopathy (BSE), mad cow disease, Kuru, Creutzfeldt-Jakob disease (CJD), Gerstmann-Sträussler-Scheinker syndrome (GSS), and/or Fatal familial insomnia (FFI).

In a further embodiment of the invention, the inoculant is a bacteria-bearing inoculant. Non-limiting examples of bacteria-bearing inoculants include the blood, plasma or serum of a person infected with bacteria, a bacterial lysate, tissue from a person infected with a bacteria, lysates of cysts or other inclusion bodies containing bacteria, a purified bacterial preparation grown in vitro, or a suspension of bacteria in saline, plasma, or another biological fluid. The bacteria may be, for example, a gram negative bacteria or a gram positive bacteria.

In a particular embodiment, the inoculant includes two or more immunogens. These immunogens may be the same type (i.e., both cancer immunogens) or different type (i.e., a cancer immunogen and a bacterial immunogen). In a particular embodiment, the inoculant contains two or more cancer immunogens. In another embodiment, the inoculant contains a viral immunogen in combination with a non-viral immunogen, e.g., a cancer immunogen or a bacterial immunogen. one of the immunogens is a viral immunogen. In a particular embodiment, the viral immunogen is a HIV immunogen. In a preferred embodiment, the inoculant contains an HIV immunogen and a cancer immunogen (e.g., a cancer cell lysate, plasma from a cancer patient).

According to one embodiment of the present invention, human blood is drawn from a patient with abnormal cell proliferation (e.g., breast cancer) or patient infected with a virus (e.g., HIV) using standard, sterile, phlebotic techniques. Preferably, patient is between 18 and 65 years of age. Preferably, donors should appear healthy, not be under the influence of drugs or alcohol, and weigh in excess of 50 kg (110 lb). The following criteria of health can also be useful: body temperature less than 37.5° C.; pulse regular (50 to 100 beats per minute); blood pressure lower than 180 mm Hg systolic and 100 mm Hg diastolic; hemoglobin greater than 12.5 g/l and hematocrit greater than 38%. The blood is then processed to produce plasma according to techniques well known to those skilled in the art.

The plasma obtained from a virus-positive patient (HIV+) or patient with abnormal cell proliferation (e.g., cancer patient) can then be used to inoculate a mammal, such as a goat. The plasma can be injected one or more times. In addition, various adjuvants can be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and corynebacterium parvurn. Such adjuvants are also well known in the art. Additional non-limiting examples of adjuvants suitable for use in the present invention are discussed further below.

The plasma and/or adjuvant can be injected in the mammal by one or more subcutaneous or intraperitoneal injections, though they can also be given intramuscularly, and/or intravenously.

The mammal can be given a sedative, for example Rompun, to facilitate handling of the mammal if necessary.

Preferably, at least 1 cc of human plasma can be administered to the mammal. For example, between 1-10 cc of the plasma can be administered to the animal subcutaneously. Alternatively, at least 1, 2, 5, 7, 10, 15, 20, 25, 30, 40 or 50 cc of plasma is administered subcutaneously, intraperitoneally intramuscularly, and/or intravenously.

In a particular embodiment, the animal is inoculated and then re-inoculated after a period of time ranging from 1, 2, 3, 4, 5, 6, 7, 8, 9, 10, 11, 12 or more weeks. The second inoculation is also known as a booster.

In one embodiment of the invention, an inoculant is not used. Rather, the serum or plasma fraction is prepared by obtaining plasma or serum from an animal, without prior inoculation.

(iii) Monitoring of Animal

The animal should preferably be monitored to indicate the patient sample with which it was injected and the date of injection. The animal should be monitored over a time period, beginning at about one week. Blood samples can be obtained from the animal during this time to measure the generated immune response. For example, the plasma from the blood sample can be tested in cell proliferation and growth inhibition assays in vitro, using, for example breast or prostate cancer cells. The mammal can be a goat. The period of time for generating an immune response can vary. For example, the period of time for generating an immune response can range from 1-8 weeks or 1-9, 10, 11, 12, 13, 14, 15, 16, 17, 18, 19, 20, 21, 22, 23, 24, 25, 26, 27, 28 weeks. It is known that in goats that three weeks is a standard period of incubation for generating a sufficient immune response.

One can assess the material using the procedures of various techniques are known in the art that include, but are not limited to: ELISA and Western blot. Other types of immunoassays known in the art can be used, including but not limited to, competitive and non-competitive assay systems using techniques such as radioimmunoassays, ELISA (enzyme linked immunabsorbant assay), “sandwich” immunoassays, immunoradiometric assays, gel diffusion precipitation reactions, immunodiffusion assays, in situ immunoassays. (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc.

An ELISA is a technique that uses antigens to coat the well of plates. ELISAs involve coating the well of a multiwell, such as a 96-well, microtiter plate with the antigen, washing away antigen that did not bind the wells, adding the blood or blood product from the mammal that has been inoculated conjugated to a detectable compound such as an enzymatic substrate (e.g., horseradish peroxidase or alkaline phosphatase) to the wells and incubating for a period of time, washing away unbound or non specifically bound materials, and detecting the presence of the specifically bound blood or blood product to the antigen coating the well. Alternately, in ELISAs the blood or blood product from the mammal that has been inoculated does not have to be conjugated to a detectable compound; instead, a second antibody (which recognizes the blood or component of the blood) can be conjugated to a detectable compound and added to the well. One of skill in the art would be knowledgeable as to the parameters that can be modified to increase the signal detected as well as other variations of ELISAs known in the art. For further discussion regarding ELISAs see, e.g., Ausubel et al, eds, 1994, 10 Current Protocols in Molecular Biology, Vol. 1, John Wiley & Sons,—Inc., New York Bollag, D. M., Rozycki, M. D., and Edelstein, S. J. (1996). Protein Methods, Second Edition. New York: Wiley-Liss, 195-227.

Another useful technique is a western blot analysis. Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA. or non-fat milk), washing the membrane in washing buffer (e.g., PBS-Tween 20), applying the proposed binding protein (diluted in blocking buffer) to the membrane, washing the membrane in washing buffer, applying a secondary antibody (which recognizes the viral protein that you are assaying for) conjugated to an enzymatic substrate (e.g., horseradish peroxidase or alkaline phosphatase) or radioactive molecule (e.g. ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc, New York 30; Harlow, E. and Lane, D. (1988). Antibodies: A Laboratory Manual. Cold Spring Harbor, N.Y.: Cold Spring Harbor Laboratory Press, 471-510.

Other techniques for evaluating immune responses in mammals are known to one skilled in the art.

(iv) Removal of Blood from Inoculated Animal

To obtain the plasma from the mammal, blood has to be collected. Any means to do this which accomplishes the desired goal is suitable. It is preferable to obtain large quantities of blood from the mammal, for example 10-30 cc of blood from a rabbit or similar sized animal and higher quantities from larger animals. The blood should begin to flow immediately through the tubing to the syringe, vacutainer, or open tube/bottle. If a syringe is used, gently draw on the syringe to collect the blood, and once the syringe is full, change syringes by disconnecting from the infusion set or needle hub. (See, for example, McGuill, M. W. and Rowan, A. N., “Biological Effects of Blood Loss: Implications for Sampling Volumes and Techniques,” ILAR News, Vol. 31(4), Fall 1989, pp 5-20).

In a particular embodiment of the present invention, the blood is collected in a manner that prevents coagulation in order to obtain plasma not serum. A variety of methods are known in the art for preventing coagulation of drawn blood, and include, without limitation, collecting the blood in tubes or other types of collecting means that have been treated with an anticoagulant. Anticoagulant coated test tubes of this type are widely available commercially. Suitable anticoagulants include, but are not limited to EDTA, heparin, citrate or oxalate. Tube inversions allow proper mixing of anticoagulant additives and blood. Alternatively, a syringe and the infusion set tubing used in harvesting the blood can be filled with anticoagulant to aid in the harvesting of the plasma. Alternatively, the blood can be collected into a vacutainer or bottle that has been treated with anticoagulant. Alternatively, anticoagulants can be added to the plasma component of the blood after the cellular elements have been removed, for example by centrifugation as described below.

It is has been observed that plasma may not remain anticoagulated over time (i.e., it may clot to produce serum) unless proper techniques are utilized. Techniques for preventing plasma instability are known to those skilled in the art.

In one embodiment of the present invention, the composition is a serum fraction. When serum is desired, as opposed to plasma, the blood should be collected in a way that permits coagulation. Blood will naturally coagulate in a collection tube as coagulation factors become activated upon contact with a negative surface (“contact activation”). The time required to clot plasma may vary, and may range from less than a minute to more than an hour. Alternatively, the clotting process can be accelerated by the addition of a clotting activator to the tube or other container used to collect blood. Non-limiting examples of suitable clotting activators include calcium or silica particles.

The animal can be sedated. For example, 0.5 cc Rompun can be used to sedate, for example, a goat. In another example, Torbugesic (butorphanol; 1 mg/kg) and acepromazine maleate (1 mg/kg) can be used to sedate, for example, a rabbit. After the animal is sedated, the blood can be collected. One way to remove blood from an animal is to cannulate an artery, for example the external jugular artery. The mammal can be a goat and for a goat, an at least 18 gauge needle can be used to extract at least 150 cc of blood, preferable between 200-400 cc of blood. In another example, a needle, at least 21 gauges, is connected to an infusion device, such as an E-Z infusion set, to a syringe, for example, at least a 10 or 20 cc syringe.

As the blood is collected, it is preferably stored in a cooled environment, for example on ice or in a refrigerator or freezer.

(v) Treatment of Conditioned Blood to Form Plasma or Serum

The following description describes one way in which the blood can be treated to form the plasma or serum.

In one embodiment, plasma is separated from blood by centrifugation. Centrifugation speeds and times are known to those skilled in the art, and may depend, for example, upon the type of tube used for blood collection. The specific gravity ranges for red cells are sufficiently different to enable isolation by centrifugation. Plasma is then obtained from the appropriate fragment.

In one embodiment of the present invention, the plasma can be repeatedly centrifuged to minimize the number of residual cells in the plasma fraction.

In another embodiment, serum is separated from clotted blood by centrifugation. Certain types of tubes known to those in the art may facilitate the separation process. For example, tubes containing a gel substance such that when the tube is centrifuged the cells go below the gel while the serum remains above.

(vi) Treatment of Plasma or Serum to Form a High Molecular Weight Depleted Fraction

The anti-abnormal cell proliferation activity of the plasma or serum is then isolated from one or more of the various high molecular weight proteins (e.g., immunoglobulins, albumin) or biological agents present in unprocessed serum or plasma.

Plasma contains a mixture of hundreds of different kinds of proteins. (For a review, see Turner, M. W., and Hulme, B. (1970) The Plasma Proteins: An Introduction, Pitman Medical & Scientific Publishing Co., Ltd., London). Serum differs from plasma in that the clotting proteins (i.e., fibronectin) have been removed. The protein content of serum is approximately 60-80 mg/ml). The majority of serum protein is represented by a few, very abundant high molecular weight (HMW) proteins. Common high molecular weight proteins include, for example, immunoglobulins, albumin, transferrin, haptoglobin and lipoproteins.

Immunoglobulins (antibodies) are globular glycoproteins found in body fluids such as serum or on B cells where they act as antigen receptors. Immunoglobulins represent 10-25% of all serum proteins. They range in molecular weight from approximately 150,000-970,000 daltons. The five major classes of immunoglobulins (IgA, IgG, IgM, IgD and IgE), are distinguished by differences in the C regions of H chains of the molecule. They differ in size, charge, amino acid composition and carbohydrate content.

IgG is the dominant immunoglobulin (70-75%) in extracellular fluids like serum and has a molecular weight of approximately 150,000 daltons. IgM is the largest immunoglobulin, and has a molecular weight of 900,000 daltons. It represents approximately 10% of the total immunoglobulin pool. IgA concentrates in body fluids such as tears, saliva, and the secretions of the respiratory and gastrointestinal tracts. IgD accounts for less than 1% of the plasma immunoglobulins, and is almost exclusively found inserted into the membrane of B cells. IgE is normally present in only trace amounts, but it is responsible for the symptoms of allergy.

Albumin is a highly-water soluble protein with a molecular weight of approximately 66,000 Da. (For a review, see Peters T., Jr. All about Albumin: Biochemistry, Genetics, and Medical Applications Academic Press, San Diego, 1996) It is the most abundant protein in human blood, representing more than 55% of total serum proteins. It plays a role in the osmotic pressure of the plasma, and also functions as a carrier for hormones, enzymes, fatty acids, and metal ions. The average concentration of albumin in human serum is 4.0-4.8 g/100 ml.

Transferrin is a metal-binding glycoprotein with a molecular weight of approximately 80,000 daltons. (For a review, see Huebers H A and Finch C A. Physiological Reviews (1987) 67: 520). The primary function of transferrin is the transport of iron in plasma. It is also known as siderophilin.

Haptoglobin is a 100,000 dalton glycoprotein. It removes free hemoglobin from the circulation of vertebrates which binds free hemoglobin, preventing loss in the urine. Other diverse properties of human haptoglobin have been observed (see, e.g., Oh S K et al. J. Leuko. Biol., (1990) 47: 142-148; Cid M C et al. J. Clin. Invest. (1993) 91: 977-985).

Lipoproteins are lipid-protein complexes which permit the transport of otherwise insoluble lipids through the blood stream. The major serum lipoproteins include chylomicrons, very low density lipoproteins (VLDL), low density lipoproteins (LDL), intermediate-density lipoproteins (IDL), and high-density lipoproteins (HDL).

Low molecular weight proteins found in plasma and serum include cytokines, chemokines, peptide hormones, as well as proteolytic fragments of large proteins. Cytokines and growth factors are typically between 6 and 50 kD, and more commonly between 10 and 30 kD. The molecular weight of various low molecular weight proteins commonly found in human serum is detailed in the commonly available BioSource catalog.

In a particular embodiment, the present invention is composition useful for the treatment or prevention of abnormal cell proliferation which is a plasma or serum fraction derived from a mammal exposed to an inoculant, which fraction has been depleted of one or more high molecular weight proteins (e.g., immunoglobulin).

In another particular embodiment, the invention is a composition useful for the treatment or prevention of abnormal cell proliferation which is a plasma or serum fraction derived from a mammal exposed to an inoculant, which has been depleted of two or more high molecular weight proteins (e.g., immunoglobulin and albumin).

Proteins can be separated from plasma or serum by fractionation. Fractionation strategies can vary in specificity, from very general to highly specific for a particular activity of interest. Fraction methods can be used alone or in combination. The goal of fractionation is to obtain a fraction enriched for an activity of interest. In the present invention, the activity of interest is believed to reside in a fraction of plasma or serum depleted of one or more high molecular weight proteins such as albumins and immunoglobulins (e.g., IgG and IgM). Put another way, the activity of interest is believed to be a low molecular weight protein or biological agent.

In one embodiment, the desired product of fractionation is a fraction which has been enriched for proteins or biological agents between approximately 6 and approximately 50 kD.

In a particular embodiment, the fraction has been enriched for proteins or biological agents between approximately 6 and approximately 20 kD, approximately 6 and approximately 14 kD, or approximately 6 and approximately 10 kD.

In another embodiment, the desired product of fractionation is a fraction which has been enriched for proteins or biological agents between approximately 30 and approximately 50 kD.

In a particular embodiment, the fraction is enriched for proteins between approximately 30 and approximately 40 kD.

In yet another embodiment of the present invention, the desired product of fractionation is a fraction enriched for proteins and biological agents between approximately 15 and approximately 25 kD.

In a still further embodiment of the present invention, the desired product of fractionation is fraction n enriched for proteins and biological agents between approximately 20 and approximately 25 kD.

In a particular embodiment, desired product of fractionation is a fraction enriched for a protein(s) or biological agent(s) with a molecular weight of approximately 12.2 kD.

In another particular embodiment, the desired product of fractionation is fraction enriched for a protein(s) or biological agent(s) with a molecular weight of approximately 14.1 kD.

In a further particular embodiment, the desired product of fractionation is a fraction enriched for a protein(s) or biological agent(s) with a molecular weight of approximately 28.6 kD In yet another particular embodiment, the desired product of fractionation is a fraction enriched for a protein(s) or biological agent(s) with a molecular weight of approximately 29 kD.

In a still further particular embodiment, the desired product of fractionation is a fraction enriched for a protein(s) or biological agent(s) with a molecular weight of approximately 30.1 kD.

In another particular embodiment, the desired product of fractionation is a fraction enriched for a protein(s) or biological agent(s) with a molecular weight of approximately 49.4 kD.

In another embodiment of the present invention, the desired product of fractionation is a fraction enriched for a protein(s) or biological agent(s) with a molecular weight of approximately 53 kD.

It may require multiple fractionation steps to isolate the anti-abnormal cell proliferation activity from one or more of the high-molecular weight proteins or biological agents. Specifically, the anti-abnormal cell proliferation activity may initially fractionate with the high molecular weight proteins, which high molecular weight fraction must then be further processed to isolate the anti-abnormal cell proliferation activity from the high molecular weigh proteins. Alternatively, the anti-abnormal cell proliferation activity may fractionate from the high molecular weight protein or proteins in a single fractionation step. Or, some of the activity of interest may initially fractionate with the high molecular weight protein fraction, while some additional portion of the activity may remain in the low molecular weight fraction.

A wide variety of methods are available to fractionate plasma or serum to isolate proteins. Such methods can be broadly divided into those which divide the protein between two phases (e.g., a solid and liquid) and those which separate proteins by different rates of movement through a material, such as a chromatographic column or electrophoresis gel. Any method capable of achieving the desired result is considered suitable for use in the present invention. These methods can be used alone, or in combination. Fractionation can involve a single step, or multiple steps.

Fractional precipitation can be used to deplete the initial serum or plasma fraction of high molecular weight proteins, such as immunoglobulins and albumins. Non-limiting examples of fractional precipitation methods include solvent, salt, isolectric, hydrophilic polymer and heat precipitation. All fractional precipitation methods rely on bringing protein out of solution by altering the medium to reduce its solubility. Once insoluble, the protein can be separated form the mixture by centrifugation or filtration. Organic solvent precipitation methods are suitable for use in the method of the present invention. Addition of the solvent results in a decrease in the dielectric constant of the medium, which produces a decrease in protein solubility. Solvents may include, for example, 2 methyl-2,4-pentane diol (MPD), Dimethyl Sulfoxide (DMSO) and ethanol. In a particular embodiment, cold alcohol fractionation or ethanol fractionation, also known as the Cohn-Oncley method, is used (Cohn E J et al. J Am Chem Soc 1946; 68: 459-75) This method involves the precipitation of proteins under varying conditions of ethanol and pH conditions.

A variety of cold ethanol fractionation methods are known in the art for isolating albumin and immunoglobulin from plasma (See e.g., Cohn E J et al. J Am Chem Soc (1946) 68: 459-75; Hink J H et al. Vox Sang (1957) 2: 174-86; Kistler P et al. Vox Sang (1962) 7: 414-24). The Cohn and Kitler methods are compared in More J E et al. In: Harris J R, ed. Blood Separation & Plasma Fractionation. New York: Wiley, 1991; 261-306). Coagulation factors are removed as cryoprecipitate on initial thawing of the plasma before cold ethanol fractionation. With either method, an initial low ethanol precipitation stage removes the fibrinogen from the source plasma. Immunoglobulins are precipitated by raising the ethanol concentration to 25% at pH 6.9 for the Cohn method or 19% at pH 5.85 for the Kistler and Nitschmann method, while albumin remains in solution. Albumin is then isolated from the majority of the other plasma contaminants (mainly alpha and beta globulins), which are precipitated by the further addition of ethanol to a final ethanol concentration of 40%. This is carried out in two stages in the Cohn process but as a single step in the Kistler and Nitschmann method. In a final step, the albumin is itself precipitated near its isoelectric point. In an alternate approach to solvent precipitation, unwanted proteins in a mixture might be specifically inactivated and denatured by an organic solvent, thus allowing the contaminating protein to be removed.

Proteins can also be separated from plasma or serum by salt precipitation. Protein solubility is a function of the physiochemical nature of the proteins, pH temperature and the concentration of the salt used. It also depends on whether the salt is Kosomtropic (stabilizes water structure) or Chaotropic (disrupts water structure). Many types of salts (e.g., ammonium sulfate) can be employed to effect protein separation and purification. Ammonium sulfate is common used because of it is highly soluble, relatively inexpensive and generally preserves protein function. Using the appropriate concentration range of the given salt, a protein of interest can be preferentially isolated from a protein mixture. According to this method, increasing amounts of ammonium sulfate are added to give a certain percentage saturated, followed by a period of time to permit proteins to precipitate, and a centrifugation step to collect the precipitate.

In one embodiment of the present invention, ammonium sulfate precipitation is used to isolate the anti-abnormal cell proliferation activity of the plasma or serum from one or more high-molecular weight proteins or biological agents present in the unfractionated plasma or serum. In one embodiment, a single ammonium sulfate precipitation step is sufficient to isolate the anti-abnormal cell proliferation activity of the plasma or serum from one or more high-molecular weight proteins or biological agents present in the unfractionated plasma or serum. In another embodiment, ammonium sulfate precipitation is used to in combination with one or more additional fractionation steps or methods, either the same or different, to isolate the anti-abnormal cell proliferation activity of the plasma or serum from one or more high-molecular weight proteins or biological agents present in the unfractionated plasma or serum. For example, sequential ammonium sulfate precipitation (“cuts”) may be used.

In a particular embodiment, ammonium sulfate precipitation is used to isolate the anti-abnormal cell proliferation activity of the plasma or serum from immunoglobulin present in the unfractionated plasma or serum. These immunoglobulin present may include IgG, IgM or both IgG or IgM.

Hydrophilic polymers such as polyethylene glycol (PEG) can also be used to precipitate proteins according to the present invention. PEG varies in chain lengths from average mol wt 1000 to 40000. Those of higher molecular weight are frequently useful in concentration schemes, with the most common PEG6000.

Isoelectric precipitation can also be used to fractionate proteins in the present invention. In general, proteins are positively charged at a low pH and negatively charged at a high pH. A protein is the least soluble when the pH of the solution is at its isoelectric point, i.e., the pH at which a protein molecule has a net charge of zero.

Heat precipitation also permits isolation of proteins according to the present invention. This method is typically used to remove contaminating proteins from a protein-containing solution. The stability of different proteins at elevated temperature varies, and if the desired protein has a greater heat stability than contaminating proteins, incubation at elevated temperatures (e.g., 45-70° C.) for a period of (i.e., varying from a few minutes to a few hours) produces precipitation of the unwanted proteins.

Dialysis and ultrafiltration can also be used to provide low-resolution protein fractionation. In dialysis, the protein sample is enclosed in a bag consisting of a semipermeable membrane (made of cellulose) and exposed to a large volume of a desired buffer. The low-molecular weight compounds (buffering agents, salts) pass freely through the membrane pores whereas the protein is retained. In ultrafiltration methods, the pores are generally larger and allow smaller proteins to pass through. Ultrafiltration typically employs pressure to force the sample through. Membranes with various molecular weight cutoffs are commercially available (i.e., from less than 10 to more than 100 kDa). Centrifugal ultrafiltration has also been used to deplete serum of large, highly abundant proteins such as albumin. (Tirumalai R S et al. Molecular & Cellular Proteomics (2003) 2:1096-1103.

Chromatographic fractionation of plasma or serum can also be used to isolate proteins from serum or plasma according to the present invention. In general, chromatography refers to any of a number of methods in which solutes are fractionated by partitioning between a mobile or buffer and an immobile or matrix, phase. Column chromatography is one type, and involves passing the starting material through a column which is constantly being washed through with a suitable buffer. As the protein enters the column, it interacts with the matrix of the column which can take many forms. Types of chromatography suitable for use in the present invention include, without limitation, gel filtration, ion exchange, affinity,

(i) Gel filtration chromatography, also known as molecular exclusion or gel permeation chromatography, separates molecules on the basis of size. In this method, the stationary phase is a gel matrix with a well-defined range of pore sizes is used. Large proteins do not enter the pores of the chromatographic matrix, but pass through, into the interstitial space between the matrix beads; this space is also known as the void volume, V0. These large proteins migrate more rapidly than small molecules which diffuse into and back out of the resin and consequently are partly trapped and fall behind. Proteins of intermediate size will penetrate to varying degrees into the beads and thus are separated from each other on the basis of their size. Low-pressure gel beads are capable of separating molecules from a molecular weight of a few hundred to multimeric proteins weighing in the millions range. These gel filtration resins are made from a variety of materials, including dextran, agarose, and polyacrylamide and are available in various pore sizes. Commercial gels include Bio-Gel (Bio-Rad) and Sephadex/Sepharose (Amersham Pharmacia Biotech).

In one embodiment of the present invention, gel filtration is used to isolate the anti-abnormal cell proliferation activity of the plasma or serum from one or more high-molecular weight proteins or biological agents present in the unfractionated plasma or serum. High molecular weight proteins, including immunoglobulins and serum albumins, typically fractionate in the first eluted fractions to come off of the column. The lower molecular weight proteins, such as cytokines, typically come off of the column in the latter fractions.

In one embodiment, a single gel filtration step is used to isolate the anti-abnormal cell proliferation activity of the plasma or serum from one or more high-molecular weight proteins or biological agents present in the unfractionated plasma or serum. In another embodiment, a gel filtration step is used to in combination with one or more additional fractionation steps or methods, either the same or different, to isolate the anti-abnormal cell proliferation activity of the plasma or serum from one or more high-molecular weight proteins present in the unfractionated plasma or serum.

(ii) Ion exchange chromatography involves the use of a stationary phase matrix with covalently linked anions or cations. Solute ions of the opposite charge in the mobile liquid phase are attracted to the resin by electrostatic forces. Under specific starting conditions of buffer, pH, and ionic strength, the net charge on the protein of interest can be manipulated to interact with the matrix. These conditions are well known to those in the art. IEC media are available in differing charges, pore sizes, and support strengths (i.e., low-pressure to high-pressure tolerant). Commercial sources of EC media include Amersham-Pharmacia, Bio-Rad, Dionex, Hewlett Packard, Merck, Perseptive Biosystems, and TosoHaas, among others.

In one embodiment of the present invention, the anti-abnormal cell proliferation activity is isolated from one or more high molecular weight proteins present in the initial plasma or serum sample using ion exchange chromatography. In one embodiment, the anti-abnormal cell proliferation activity is isolated from one or more high molecular weight proteins or biological agents present in the initial plasma or serum sample using a single ion exchange chromatography step. In another embodiment, the anti-abnormal cell proliferation activity isolated from one or more high molecular weight proteins or biological agents present in the initial plasma or serum sample using an ion exchange chromatography step in combination with one or more additional fractionation steps or methods, either the same or different.

In a particular embodiment, the anti-abnormal cell proliferation activity is isolated from immunoglobulins (i.e., IgG, IgM or both) using ion exchange chromatography. In another embodiment, anti-abnormal cell proliferation activity is isolated from two or more high molecular weight proteins present in the initial serum sample. In a particular embodiment, the anti-abnormal cell proliferation activity is isolated from immunoglobulins and albumin.

(iii) Affinity chromatography involves the specific interaction between one molecule in the sample and a second molecule immobilized on a stationary phase. Proteins can be used to isolate antibodies and vice versa. The affinity may be to a specific protein or a group of proteins. If the protein to be fractionated isolated is a gamma globulin, protein A is often used. Serum which contains the secreted antibodies is put through the affinity column, and the antibodies bind to the protein A attached to the column gel. Other ligands suitable for use in isolating components of blood include heparin (clotting-factor proteins), lectins (glycoproteins), antibodies (unique antigens), and enzyme inhibitors or cofactors (enzymes). For example, VLDL and LDL can be removed from a sample using antibody-based affinity chromatography (also known as immunoabsorption). Sources include, for example, Amersham-Pharmacia and Bio-Rad.

In a particular embodiment of the present invention, the anti-abnormal cell proliferation activity is isolated from one or more high-molecular weight proteins or biological agents in the initial serum or plasma sample using affinity chromatography. Affinity chromatography may be used alone or in combination with other fractionation steps or methods detailed herein. In a particular embodiment, a protein G affinity column is used to deplete the sample of IgG to further isolate the anti-abnormal cell proliferation activity.

Methods of isolating albumin from serum involving chromatographic adsorbents and immunoaffinity methods have been reported (Sato A K et al., Biotechnol. Prog. (2002) 18, 182-192; Dockal M et al. J. Biol. Chem. (1999) 274, 29303-29310; Rothemund D et al. Proteomics (2003) 3, 279-287).

Recent advances in the study of the human proteome have led to the development of techniques to remove high abundance proteins from serum in order to isolate LMW proteins (see generally, Tirumalai R S et al. Molecular & Cellular Proteomics (2003) 2:1096-1103). Several of these methods are designed to retain LMW proteins which would otherwise be lost in the fractionation because they tend to bind to HMW proteins. These methods are considered suitable for use in isolating the anti-abnormal cell proliferation activity of the plasma or serum of a mammal exposed to an inoculant from the high molecular weight proteins present therein, including, for example, immunoglobulins and albumins.

Commercial affinity depletion products are available to separate albumin and immunoglobulins from serum or plasma. For example, the ProteoExtract™ Albumin/IgG Removal Kit (CalbioChem) provides highly specific and efficient depletion of albumin and IgG from plasma or serum. Depletion of albumin and IgG removes up to 75% of total serum proteins. Applied BioSystems also manufactures affinity depletion cartridges for the removal of albumin (POROS® Anti-HAS support) and IgG (POROS® Protein G cartridge) from serum (Application Note: Protemics. Affinity Depletion Cartridges for Removal of Human Serum Albumin and Immunoglobulins from Human Serum. Applied Biosystems). Using these products, greater than 99% of IgG and albumin can be removed from serum. These commercial affinity depletion products are considered suitable for use in preparing the plasma or serum fraction of the present invention.

Polyclonal antibodies can be used to deplete plasma or serum of high molecular proteins in a single step, including albumin, IgG, IgA, haptoglobin, transferrin, and antitrypsin, using liquid chromatography. Commercial sources of multi affinity removal systems include Agilent. This technique removes 85-90% of the high abundance proteins from serum. These multi-affinity removal systems are considered suitable for use in preparing the plasma or serum fraction of the present invention.

In one embodiment of the present invention, a single fractionation is sufficient to isolate the anti-abnormal cell proliferation activity of the plasma or serum from one or more high-molecular weight proteins or biological agents present in the initial, unprocessed plasma or serum sample. Alternatively, two or more fractionation steps can be combined. Each step may be the same basic technique (e.g., multiple ammonium sulfate precipitations) or different (e.g., precipitation with cold ethanol in combination with chromatography or heat precipitation). Any method or combination of methods suitable for isolating the anti-abnormal cell proliferation activity in the initial plasma or serum sample from immunoglobulins and other high molecular weight proteins is considered suitable for use in the present invention.

In a particular embodiment, the anti-abnormal cell proliferation activity is isolated from immunoglobulins and albumin present in the initial plasma or serum sample by sequential ammonium sulfate precipitation in combination with DEAE column chromatography.

According to one embodiment of the present invention, proteins or biological agents with a molecular weight of greater than approximately 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 67, 68, 69, 70, 71, 72, 73, 74, 75, 76, 77, 78, 79, 80, 81, 82, 83, 84, 85, 86, 87, 88. 89, 90, 91, 92, 93, 94, 95, 96, 97, 98, 99, 100, 101, 102, 103, 104, 105, 106, 107, 108 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 or 145 kD or above are depleted from the initial serum or plasma sample to facilitate isolation of the anti-abnormal cell proliferation activity.

In a particular embodiment, 10-20%, 20-30%. 30-40%, 40-50%, 50-60%, 60-70%, 70-80%, 80-90% or 90-100% of the proteins of biological agents with a molecular weight of greater than approximately 10, 11, 12, 13, 14, 15, 16, 17, 18, 19, 20, 21, 22, 23, 24, 25, 26, 27, 28, 29, 30, 31, 32, 33, 34, 35, 36, 37; 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 67, 68, 69, 70, 71, 72, 73, 74, 75, 76, 77, 78, 79, 80, 81, 82, 83, 84, 85, 86, 87, 88. 89, 90, 91, 92, 93, 94, 95, 96, 97, 98, 99, 100, 101, 102, 103, 104, 105, 106, 107, 108 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 or 145 kD or above are depleted from the initial serum or plasma sample to facilitate isolation of the anti-abnormal cell proliferation activity.

In a particular embodiment, the serum or plasma is depleted of proteins or biological agents with a molecular weight greater than approximately 15 kD to facilitate isolation of the anti-abnormal cell proliferation activity.

In another embodiment, the serum or plasma is depleted of proteins or biological agents with a molecular weight greater than approximately 20 kD to facilitate isolation of the anti-abnormal cell proliferation activity.

In a further embodiment, the serum or plasma is depleted of proteins or biological agents with a molecular weight greater than approximately 25 kD to facilitate isolation of the anti-abnormal cell proliferation activity.

In a preferred embodiment, the serum or plasma is depleted of proteins or biological agents with a molecular weight greater than approximately 30 kD to facilitate isolation of the anti-abnormal cell proliferation activity.

In a further embodiment, the serum or plasma is depleted of proteins or biological agents with a molecular weight greater than approximately 35 kD to facilitate isolation of the anti-abnormal cell proliferation activity.

In another embodiment, the serum or plasma is depleted of proteins or biological agents with a molecular weight greater than approximately 40 kD to facilitate isolation of the anti-abnormal cell proliferation activity.

In a further embodiment, the serum or plasma is depleted of proteins or biological agents with a molecular weight greater than approximately 45 kD to facilitate isolation of the anti-abnormal cell proliferation activity.

In a further embodiment, the serum or plasma is depleted of proteins or biological agents with a molecular weight greater than approximately 50 kD to facilitate isolation of the anti-abnormal cell proliferation activity.

In yet another embodiment, the serum or plasma is depleted of proteins or biological agents with a molecular weight of greater than approximately 90 kD to facilitate isolation of the anti-abnormal cell proliferation activity.

The term “depletion” is used to indicate a reduction in the amount of a compound(s) or molecule(s) (e.g., high molecular weights proteins) in a given sample after the sample is treated according to the method of the present invention. In one embodiment of the present invention, the sample is depleted of 100%, 99%, 98%, 97%, 96%, 95%, 94%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 56%, 54%, 53%, 52%, 51%, 50%, 49-45%, 44-40%, 39-35%, 34-30%, 29-20%, 19-10%, 10%-5%, 5%-1% of the one or more high molecular weight proteins or biological agents present in the initial plasma or serum sample (e.g., immunoglobulin, serum albumin, transferrin, haptoglobin or lipoproteins).

In one embodiment of the present invention, the plasma or serum is depleted of substantially all high molecular weight proteins. In a particular embodiment, the plasma or serum is depleted of substantially all proteins with a molecular weight greater than approximately 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 67, 68, 69, 70, 71, 72, 73, 74, 75, 76, 77, 78, 79, 80, 81, 82, 83, 84, 85, 86, 87, 88.89, 90, 91, 92, 93, 94, 95, 96, 97, 98, 99, 100, 101, 102, 103, 104, 105, 106, 107, 108 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 or 145 kD

As noted above, while the initial sample may be depleted of one or more high molecular weight proteins or biological agents to facilitate isolation of the anti-abnormal cell proliferation activity, the anti-abnormal cell proliferation activity may initially co-fractionation with one or more of the high molecular weight proteins or biological agents, such that the high molecular weight fraction must be further fractionated or processed to isolate the anti-abnormal cell proliferation activity present therein.

The anti-abnormal cell proliferation activity of the fractionated serum or plasma can be compared to the anti-abnormal cell proliferation activity of the starting sample (i.e, the unfractioanted serum or plasma) at any point during the fractionation procedure. The baseline activity of the unfracitonated sample can be calculated using methods known in the art. For example, one can measure the protein concentration of the serum or plasma sample using Bradford or Lowery based methods for determining protein concentration. Then, the activity of the faction can be established using an in vitro assay (e.g., activity in a breast cancer proliferation assay). A unit of activity can be assigned to the fraction based on the amount of protein needed to achieve a 50% inhibition (IC50). This unit can be used as a baseline to track the fold purification obtained during the fractionation process.

Transmission of infectious disease (i.e., by viruses, bacteria or parasites) remains a concern in the use of any blood or blood product such as plasma or serum. In a further embodiment of the present invention, the blood or plasma can be sterilized prior to in vivo use. Any suitable method can be used to achieve sterilization as long as the method does not alter the product in such a way as to diminish its efficacy. Non-limiting examples of sterilization techniques suitable for use with the present invention include chemicals, heat, ultraviolet radiation and photosensitizing dyes. The plasma can also be filtered to achieve sterilization. Recent advances and new strategies for the inactivation and removal of infectious agents are contemplated for use in the present invention.

In one embodiment of the present invention, the plasma is separated from blood and sterilized by repeated centrifugation and filtration. For example, the plasma is spun at approximately 32,000 rpm on a standard centrifuge. The resultant supernatant can then be transferred, preferably under sterile conditions using sterile techniques, and then suction filtered through a 0.5 micron filter. During this preparation, the sample can be kept on ice between the centrifugation and filtration steps. The plasma can then be passed over a filter, for example a filter with at least 0.2 micron pores, and then placed in an ultracentrifuge, preferably non-refrigerated, to spin at approximately 90,000 rpm for at least 20 minutes. The supernatant can then be placed in containers, preferable sterile, in an ultracentrifuge, preferably non-refrigerated, to spin at least 150,000 rpm for at least 20 minutes. After the centrifugation, the supernatant can be passed through an anhydrous filter. The plasma can be repeatedly filtered, preferably through a 0.2 micron filter and a smaller filter, such as a 0.1 micron filter. Passage through a 0.1 micron filter allows for the plasma to be deemed sterile.

(vi) Storage and Testing of the Plasma or Serum Fraction.

The resulting plasma or serum preparation can be placed in small aliquots (e.g., between 2-10 cc each) and stored for later use. Proper storage conditions for plasma and serum with respect to temperature and time are well known to those skilled in the art. For example, test tubes containing small aliquots of the plasma or serum fraction can be stored at −70° C., for at least 48 hours.

After a suitable time has passed for the samples to be stored, such as 48 hours, individual aliquots can be brought to room temperature for sterility testing. For example, the sample can be cultured under both anaerobic and aerobic conditions to test for contamination. If the cultures are negative, the remaining aliquots of can then are administered to a patient.

(vii) Administration of Plasma or Serum Fraction to Patient in Need Thereof.

The plasma or serum fraction can be administered to a patient in need thereof through any means provided in this application (See Pharmaceutical Compositions below). In one embodiment, the patient can receive a therapeutically effective dosage, preferably between 2-10 cc if administered subcutaneously, and treatment duration can vary based on the severity of the disease.

B. Disorders of Abnormal Cell Proliferation

The plasma or serum fraction of the present invention can be used to treat or prevent abnormal cell proliferation, including diseases and disorders associated with abnormal cell proliferation. Disorders of abnormal cell proliferation incude cancer as well as other abnormal cell proliferation-associated diseases, as described above and in the section that follows. The composition can be used to treat or prevent any of the disorders of abnormal cell proliferation identified in the Background of the Invention, as well as the following non-limiting examples of such disorders that appear below.

(i) Cancers

Cancer is a group of diseases that are characterized by unregulated or abnormal cell proliferation. Cancers are commonly classified as carcinomas, sarcomas, lymphomas or leukemias based on the tissue type from which they arise. Different types of carcinomas, sarcomas, lymphomas, and leukemias are typically named using different prefixes represents the cell type including adeno-(gland), chondro-(cartililege), erythro-(red blood cell); hemangio-(blood vessel), hepato-(liver), lipo-(fat), lympho-(lymphocyte), melano-(pigment cell), myelo-(bone marrow), myo-(muscle) and osteo-(bone).

Carcinomas that can be treated or prevented with the plasma or serum fraction of the present invention are tumors arising from epithelial tissue, such as glands, breast, skin, and linings of the urogenital, digestive, and respiratory systems. Lung, cancer and prostate cancers can be treated or prevented. Breast cancers that can be treated or prevented with the composition of the present invention include both invasive (e.g., infiltrating ductal carcinoma, infiltrating lobular carcinoma infiltrating ductal & lobular carcinoma, medullary carcinoma, mucinous (colloid) carcinoma, comedocarcinoma, paget's disease, papillary carcinoma, tubular carcinoma, adenocarcinoma (NOS) and carcinoma (NOS)) and non-invasive carcinomas (e.g., intraductal carcinoma, lobular carcinoma in situ (LCIS), intraductal & LCIS, papillary carcinoma, comedocarcinoma). The present invention can also be used to treat or prevent metastatic breast cancer. Non-limiting examples of metastatic breast cancer include bone, lung and liver cancer.

Prostate cancers that can be treated or prevented with the composition of the present invention include localized, regional and metastatic prostate cancer. Localized prostate cancers include A1-A2, T1a-T1b, T1c, B0-B2 or T2a-T2c. C₁-C₂ or T3a-N0, prostate cancers extending beyond the prostate but without lymph node involvement, are also contemplated. Regional prostate cancers include D1 or N1-M0, while metastatic prostate cancers include D2 or M1. Metastatic prostate cancers include bone and brain cancers.

Other cancers that can be treated or prevented with the composition of the present invention include, but are not limited to, cancers of the cancers include those of the bowel, bladder, brain, cervix, colon, rectum, esophagus, eye, head and neck, liver, kidney, larynx, lung, skin, ovary, pancreas, pituitary gland, stomach, testicles, thymus, thyroid, uterus, and vagina as well as adrenocortical cancer, carcinoid tumors, endocrine cancers, endometrial cancer, gastric cancer, gestational trophoblastic tumors, islet cell cancer, and mesothelioma.

Lymphomas that can be treated or prevented with the plasma or serum fraction include are tumors arising from the lymph or spleen. lymph nodes and spleen, causing excessive production of lymphocytes, including both Hodgkin's disease and Non-Non-Hodgkin's lymphoma. The term “Hodgkin's Disease” is intended to include diseases classified as such by the REAL and World Health Organization (WHO) classifications known to those of skill in the art, including classical Hodgkin's disease (i.e., nodular sclerosis, mixed cellularity, lymphocyte depletion or lymphocyte rich) or lymphocyte predominance Hodgkin's disease. The term “Non-Hodgkin's lymphoma” is used to refer 30 lymphomas classified by WHO (Harris N L, Jaffe E S, Kiebold J, Flandrin G, Muller-Hermelink H K, Vardiman J. Lymphoma classification-from controversy to consensus: the REAL and WHO Classification of lymphoid neoplasms. Ann Oncol. 2000; 11(suppl 1):S3-S10), including but not limited to:

B-cell non-Hodgkin's lymphomas such as small lymphocytic lymphoma (SLL/CLL), mantle cell lymphoma (MCL), follicular lymphoma marginal zone lymphoma (MZL), extranodal (MALT lymphoma), nodal (Monocytoid B-cell lymphoma), splenic, diffuse large cell lymphoma, burkitt's lymphoma and lymphoblastic lymphoma.

T-cell non-Hodgkin's lymphoma's such as lymphoblastic lymphomas, peripheral T-cell lymphoma. Hepatosplenic gamma-delta T-cell lymphoma, subcutaneous panniculitis-like lymphoma, angioimmunoblastic T-cell lymphoma (AILD), extranodal NK/T cell lymphoma, nasal type, intestinal T-cell lymphoma (+/−enteropathy associated) (EATL), adult T-cell leukemiallymphoma (HTLV-1 associated), mycosis fungoides/Sezary syndrome, anaplastic large cell lymphoma (ALCL), including both primary cuteous and primary systemic types.

Leukemias that can be treated or prevented with the composition of the present invention include but are not limited to myeloid and lymphocytic (sometimes referred to as B or T cell leukemias) or myeloid leukemias, both chronic and acute. The myeloid leukemias include chronic myeloid leukemia (CML) and acute myeloid leukemia (AML) (i.e., acute nonlymphocytic leukemia (ANLL)). The lymphocytic leukemias include acute lymphocytic leukemia (ALL), chronic lymphocytic leukemia (CLL)(i.e., chronic granulocytic leukemia) and hairy cell leukemia (HCL).

Sarcomas that can be treated or prevented with the composition of the present invention include both bone and soft-tissue sarcomas of the muscles, tendons, fibrous tissues, fat, blood vessels nerves, and synovial tissues. Non-limiting examples include fibrosacromas, rhabdomyosarcomas, liposarcomas, synovial sarcomas, angiosacromas, neurofibrosarcomas, gastrointestinal stroma tumors, Kaposi's sacroma, Ewing's sarcoma, alveolar soft-part sarcoma, angiosarcoma, dermatofibrosarcoma protuberans, epithelioid sarcoma, extraskeletal chondrosarcoma, extraskeletal osteosarcoma, fibrosarcoma, leiomyosarcoma, liposarcoma, malignant fibrous histiocytoma, malignant hemangiopericytoma, malignant mesenchymoma, malignant schwannoma, malignant peripheral nerve sheath tumor, parosteal osteosarcoma, peripheral neuroectodermal tumors, rhabdomyosarcoma, synovial sarcoma, and sarcoma, NOS.

(ii) Other Diseases of Abnornal Cell Proliferation

Diseases of abnormal cell proliferation other than cancer can be treated or prevented with the composition of the present invention. Diseases association with the abnormal proliferation of vascular smooth muscle cells are included, including, for example, benign tumors. Non-limiting examples of benign tumors include benign bone, brain and liver tumors.

Other diseases associated with abnormal cell proliferation include, for example, atherosclerosis and restenosis. Diseases associated with abnormal proliferation of over-proliferation and accumulation of tissue mast cells are also included, such as cutaneous mastocytosis (CM) and Urticaria pigmentosa. Diseases associated with abnormal proliferation of xesangial cell proliferation are also contemplated, including but not limited to IgA nephropathy, membranoproliferative glomerulonephritis (GN), lupus nephritis and diabetic nephropathy. Rheuamatoid arthritis can be treated or prevented using the present invention.

All forms of psoriasis can be treated or prevented by the present invention, including but not limited to, plaque psoriasis, guttate psoriasis, inverse psoriasis, seborrheic psoriasis, nail psoriasis, generalized erythrodermic psoriasis (also called psoriatic exfoliative erythroderm), pustular psoriasis, and Von Zumbusch psoriasis.

The present invention can also be used to treat or prevent lymphangiomyomatosis (LAM), as well as other diseases associated with abnormal cell proliferation known to those skilled in the art.

C. Agents that can be Used in Combination and/or Alternation with the Composition of the Present Invention

The plasma or serum fraction of the present invention can be administered alone or can be administered in combination or alternation with other agents/drugs that can be used to treat or prevent abnormal cell proliferation. It can also be used alone or in combination or alternation with other agents or drugs used to treat or prevent cancer. In general, during alternation therapy, an effective dosage of each agent is administered serially, whereas in combination therapy, effective dosages of two or more agents are administered together. The dosages will depend on such factors as absorption, bio-distribution, metabolism and excretion rates for each drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. Examples of suitable dosage ranges can be found in the scientific literature and in the Physicians Desk Reference. Many examples of suitable dosage ranges for other compounds described herein are also found in public literature or can be identified using known procedures. These dosage ranges can be modified as desired to achieve a desired result.

(i) Antiproliferative Agents

The serum or plasma fraction of the present invention can also be used in combination or alternation with any of the agents or drugs for the treatment or prevention or proliferation disease treatments described in the Background of the Invention of this specification or as well as other anti-proliferative agents. Any of the antiproliferative agents listed below, or any other such agent known or discovered to exhibit an antiproliferative effect can be used in combination or alternation with the present invention to achieve a combination therapeutic effect.

Representative adjuncts include levamisole, gallium nitrate, granisetron, sargramostim strontium-89 chloride, filgrastim, pilocarpine, dexrazoxane, and ondansetron. Physicians' Desk Reference, 50th Edition, 1996.

Representative androgen inhibitors include flutamide and leuprolide acetate. Physicians' Desk Reference, 50th Edition, 1996.

Representative antibiotic derivatives include doxorubicin, bleomycin sulfate, daunorubicin, dactinomycin, and idarubicin.

Representative antiestrogens include tamoxifen citrate and analogs thereof. Physicians' Desk Reference, 50th Edition, 1996. Additional antiestrogens include nonsteroidal antiestrogens such as toremifene, droloxifene and roloxifene. Magarian et al., Current Medicinal Chemistry, 1994, Vol. 1, No. 1.

Representative antimetabolites include fluorouracil, fludarabine phosphate, floxuridine, interferon alfa-2b recombinant, methotrexate sodium, plicamycin, mercaptopurine, and thioguanine. Physicians' Desk Reference, 50th Edition, 1996.

Representative cytotoxic agents include doxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci. Physicians' Desk Reference, 50th Edition, 1996.

Representative hormones include medroxyprogesterone acetate, estradiol, megestrol acetate, octreotide acetate, diethylstilbestrol diphosphate, testolactone, and goserelin acetate. Physicians' Desk Reference, 50th Edition, 1996.

Representative immunodilators include aldesleukin. Physicians' Desk Reference, 50th Edition, 1996.

Representative nitrogen mustard derivatives include melphalan, chlorambucil, mechlorethamine, and thiotepa. Physicians' Desk Reference, 50th Edition, 1996.

Representative steroids include betamethasone sodium phosphate and betamethasone acetate. Physicians' Desk Reference, 50th Edition, 1996.

Representative antineoplastic agents include paclitaxel or doxorubicin.

Additional suitable chemotherapeutic agents include alkylating agents, antimitotic agents, plant alkaloids, biologicals, topoisomerase I inhibitors, topoisomerase II inhibitors, and synthetics. AntiCancer Agents by Mechanism, tttp://www.dtp.nci.nih.gov/docs/cancer/searches/standard_mechanism_list.html, Apr. 12, 1999; Approved Anti-Cancer Agents, http://www.ctep.info.nih.gov/handbook/HandBookText/fda_agen.htm, pages 1-7, Jun. 18, 1999; MCMP 611 Chemotherapeutic Drugs to Know, http//www.vet.purdue.edu/depts/bms/courses/mcmp611/chrx/drg2no61.html, Jun. 24, 1999; and Chemotherapy, http://www.vetmed.lsu.edu/oncology/Chemotherapy.htm, Apr. 12, 1999.

Representative alkylating agents include asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864. AntiCancer Agents by Mechanism, http://dtp.nci.nih.gov/docs/cancer/searches/standard_mechanism_list.html, Apr. 12, 1999.

Representative antimitotic agents include allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate. AntiCancer Agents by Mechanism, http://dtp.nci.nih.gov/docs/cancer/searches/standard_mechanism_list.html, Apr. 12, 1999.

Representative plant alkaloids include actinomycin D, bleomycin, L-asparaginase, idarubicin, vinblastine sulfate, vincristine sulfate, mitramycin, mitomycin, daunorubicin, VP-16-213, VM-26, navelbine and taxotere. Approved Anti-Cancer Agents, http://ctep.info.nih.gov/handbook/HandBook Text/fda_agent.htm, Jun. 18, 1999.

Representative biologicals include alpha interferon, BCG, G-CSF, GM-CSF, and interleukin-2. Approved Anti-Cancer Agents, http://ctep.info.nih.gov/handbook/HandBookText/fda_agent.htm, Jun. 18, 1999.

Representative topoisomerase I inhibitors include camptothecin, camptothecin derivatives, and morpholinodoxorubicin. AntiCancer Agents by Mechanism, http://dtp.nci.nih.gov/docs/cancer/searches/standard_mechanism_list.html, Apr. 12, 1999.

Representative topoisomerase II inhibitors include mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16. AntiCancer Agents by Mechanism, http://dtp.nci.nih.gov/docs/cancer/searches/standard_mechanism_list.html, Apr. 12, 1999.

Representative synthetics include hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium. Approved Anti-Cancer Agents, http://ctep.info.nih.gov/handbook/HandBookText/fda_agen.htm, Jun. 18, 1999.

Representative antibodies include Monoclonal antibodies directed to proliferating cells such as Rituximab (anti-CD20) for B-cell tumors and herceptin.

Drugs in clinical trials for cancer are specifically contemplated including, but not limited to: 715992 (kinesin inhibitor)(GlaxoSmithKline); Advexin (Introgen Therapeutics); AG-002037 (Pfizer); APC8024 (Dendreon); atrasentan (ABT-627); BIBH 1 (Boerhinger-Ingelheim) CCl 779 (Wyeth Pharmaceuticals); CEA Vac (Titan Pharmaceuticals); CEA-CIDE (Immunomedics) CEA-Scan (Immunomedics); Celebrex (Pharmacia); CP-547, 632 (anti-VEGF tyrosine kinase)(OSI Pharmaceuticals); CP-724-714 (anti-ErbB2[HER-2 neu] tyrosine kinase)(OSI Pharmaceuticals); CpG 7909 (Aventis Pharmaceuticals); dendritic/cancer cell fusion (Genzyme Molecular Oncology); ERA 923 (tissue-selective estrogen receptor modulator-SERM) (Ligand Pharmaceuticals); Ethyol (MedImmune Oncology); fowlpox-(6D)-TRICOMI vaccinia-(6D)-TRICOM vaccine (National Cancer Institute); G-3139 (Genta); Gemzar (Eli Lilly); Genasense (Genta); GeneVax (Centocor); GPI-0100 immune enhancer (adjuvant)(Galencia Pharmaceuticals); GTI 2040 (Lorus Therapeutics); GTI 2501 (Lorus Therapeutics); H11 (Viventia Biotech); interleukin-4 (IL-4) (National Cancer Institute); irofulven (National Cancer Institute); liquid IL-2 (Chiron); MAb antibody 3A1 (National Cancer Institute); multitargeted antifolate I (Eli Lily); Myocet (Liposome Company); oral paclitaxel (IVAX Pharmaceuticals); P53 and RAS vaccine (National Cancer Institute); PD-183805 (Pfizer); Proleukin (Chiron); ProMune (Chiron); R1550 (Antisoma); RAS peptides (National Cancer Institute); rebeccamycin analog (National Cancer Institute); recombinant human chorionic gonadotropin (r-hCG) (Serono); RSR-13 (Allos Therapeutics); RSR-13 (Eli Lilly); Targretin (Ligand Pharmaceuticals); tariquidar (QLT); Taxotere (Aventis Pharmaceuticals); TLK286 (Telik); vaccina-MUC-1 vaccine (Therion Biologics); vaccinia-MUC-1 vaccine (National Cancer Institute); Xtotax (Cell Therapeutics); Xyotax (Cell Therapeutics); Yondelis (ET-743)(Johnson & Johnson); Zarnestra (Johnson & Johnson); ZD6126 and ZD6474 (AstraZeneca); and Zoladex (AstraZeneca).

(ii) Gene Therapy

The composition of the present invention can also be used in combination or alternation with gene therapy for the treatment or prevention of abnormal cell proliferation, including cancer.

Eukaryotic cells that may be transduced with vectors (i.e., infectious viral particles or plasmids) containing a gene therapeutic, but are not limited to, primary cells, such as primary nucleated blood cells, such as leukocytes, granulocytes, monocytes, macrophages, lymphocytes (including T-lymphocytes and B-lymphocytes), totipotent stem cells, and tumor infiltrating lymphocytes (TIL cells); bone marrow cells; endothelial cells; epithelial cells; keratinocytes; stem cells; hepatocytes, including hepatocyte precursor cells; hepatocytes, including hepatocyte precursor cells; fibroblasts; mesenchymal cells; mesothelial cells; parenchymal cells, or other cells of tumor derivation.

Optionally, the vector can also contain genes that enhance the therapeutic effects of the cell. Examples of suitable genes include those that encode cytokines such as TNF, GMCSF, interleukins (interleukins 1-18), interferons (alpha, beta, gamma-interferons).

In general, a gene cannot be directly inserted into a cell. It must be delivered to the cell using a carrier known as a “vector.” The most common types of vectors used in gene therapy are viruses. Scientists use viruses because they have a unique ability to attach to or enter a cell's DNA. Viruses used as vectors in gene therapy are genetically disabled; they are unable to reproduce themselves, though they can replicate coordinately with the cellular DNA. Many gene therapy clinical trials rely on mouse retroviruses to deliver the desired gene. Other viruses used as vectors include adenoviruses, adeno-associated viruses, poxviruses and the herpes virus.

Viral vectors all induce some degree of immunological response and may have other safety risks, such as insertional mutagenesis and direct toxicity. Furthermore, large-scale production may be difficult to achieve. Therefore, in some embodiments of the invention, non-viral methods of gene transfer are used in combination or alternation with the plasma or serum fraction. These non-viral vectors may require only a small number of proteins, have a virtually infinite capacity, have no infectious or mutagenic capability, and large-scale production is possible using pharmaceutical techniques. There are at least three methods of non-viral DNA transfer, including naked DNA, liposomes and molecular conjugates.

(iii) Immunotherapy

The serum or plasma fraction of the present invention can also be used in combination or alternation with any of the immunotherapy agents or drugs for the treatment or prevention of abnormal cell proliferation disease treatments described in the Background of the Invention of this specification as well as other immunotherapeutic agents. Any of the immunotherapy agents listed below, or any other such agent known or discovered to exhibit an immunotherapeutic effect can be used in combination or alternation with the present invention to achieve a combination therapeutic effect including: cytokines such as interferon-alpha, interferon-beta, interferon-gamma, and tumor necrosis factor; monoclonal antibodies such as Rituximab (Rituxan) and trastuzumab (Herceptin); bone and marrow stem cell transplants, including twin-donors, allogenic-donors and mismatched-donors, and more particularly including non-ablative allogeneic stem cell transplantation and autologous transplantation; immunotoxins, hybrid proteins consisting of an antibody and a toxin; cancer vaccines including dendritic cell cancer vaccines.

The composition of the present invention can also be used in combination or alternation with radiation therapy, in all forms known those skilled in the art.

When used to treat leukemia, the composition of the present invention can be used in combination or alternation with tyrosine kinases such as Imatinib mesylate (Gleevec™) or other drugs known in the art for the treatment of leukemia.

(iv) Atherosclerosis Agents

When used to treat atherosclerosis or restenosis, the composition of the present invention can be used in alternation or combination with any agent or drug known for the treatment of either disease, including but not limited to:HMG-CoA reductase inhibitors including Pravastatin (Pravachol), Simvastatin (Zocor), Lovastatin (Mevacor, Altocor), Fluvastatin (Lescol), Atorvastatin (Lipitor), Rosuvastatin (Crestor); Fibric acid derivatives including Fenofibrate (Tricor) and Gemfibrozil (Lopid); Bile acid sequestrants including Cholestyramine (Questran, LoCholest, Prevalite) and Colestipol (Colestid); Antioxidants including vitamins C, E (Vita-Plus E, Softgels, Aquasol E), beta-carotene; Nicotinic acid derivatives including Niacin (Niaspan, Niacor, Slo-Niacin); other agents including but not limited to probucol, statins, aspirin, macrolide therapy, angiotensin-converting enzyme inhibitors, ACAT inhibitors, beta-blockers, atorvastatin, ticlopidine, and clopidogrel (inhibitors of platelet clumping) or other anticoagulants.

Drugs in clinical development for athersclerosis are also contemplated, including but not limited to AGI-1067 (Atherogenics), AGO-1067 (Atherogenics), Antrin (Pharmacyclics), avasimibe (ACAT inhibitor)(Pfizer), BO-653 (Chugai Pharmaceuticals), CETi-1 vaccine (AVANT Immunotherapuetics), CP-529,414 (Pfizer), SB480848 (Lp-PLA2 inhibitor) (GlaxoSmithKline), and Zithromax (Pfizer). Other clinical agents include the immunomodulatory drug DiNAC, N,N′-diacetyl-L-cystine (Astrazeneca); PPARgamma agonists (Abbott Laboratories);

(v) Rheumatoid Arthritis Agents

When used to treat rheumatoid arthritis, the composition of the present invention can be used in alternation or combination with any agent or drug known for the treatment of rheumatoid arthritic, including but not limited to: Remicade® (infliximab);methotrexate; Nonsteroidal anti-inflammatory drugs (NSAIDs) such as aspirin and ibuprofen; corticosteroid medications, such as Prednisone; Leflunomide; biologic agents such as etanercept, infliximab, adalimumab, and anakinra; celecoxib; tetracyclines; tumour necrosis factor (TNF) antagonists; nonsteroidal anti-inflammatories; cyclooxygenase-2 inhibitors; interleukin-1-receptor antagonist

Drugs in clinical investigation are contemplated, including but not limited to: 681323 (p38 alpha kinase inhibitor) (GlaxoSmithKline); 683699 (T-0047) (dual alpha 4 integrin antaginist) (GlaxoSmithKline); ABT-963 (Abbott Laboratories); AGIX-4207 (Atherogenics); alpha-L-iduronidase (Genzyme General), AMG719 (Amgen); AnergiX.RA (Corixa); anti-CD11 humanized MAb (Genentech); Arava (Aventis Pharmaceuticals); CDP 870 (Pfizer); CDP-870 (Pfizer); Celebrex (Pfizer); COX 189 (Novartis); eculizumab (Alexion Pharmaceuticals); HuMax-IL15 (Amgen); IDEC 151 (IDEC Pharmaceuticals); IDEC-151/clenoliximab (IDEC Pharmaceuticals; IL-1 trap (Rengeneron Pharmaceuticals); interleukin-1 (Regeneron Pharmaceuticals); interleukin-18 (Regeneron Pharmaceuticals); J695 (Abbott Laboratories); Oraprine (DORBioPharma); pegsunercept (soluble tumor necrosis factor-a receptor type 1)(Amgen); pralnacasan (Aventis); Prograf (Fujisawa Healthcare); r-IL-18 bp (Serono); R1487 (kinase inhibitor)(Roche); Rituxan (Genentech); SB281832 (GlaxoSmithKIine); SCIO-323 (Scio); SCIO-469 (Scio) and Vitaxin (MedImmune).

(vi) Psoriasis Agents

When used to treat psoriasis, the composition of the present invention can be used in alternation or combination with any agent or drug known for the treatment of psoriasis, including but not limited to: tar (e.g., Exorex), topical corticosteroids, topical calcipotriene (Dovonex), topical tazarotene (Tazorac), anthralin (short contact therapy), corticosteroid tape (Cordran tape), and intralesional triamcinolone; UVB phototherapy; Psoralen+UVA (PUVA) and PUVA+acitretin (Re-PUVA); Acitretin (Soriatane); Methotrexate; Cyclosporine (Neoral); other immune inhibitors such as Mycophenolate mofetil, Hydroxyurea, and Leflunomide; other treatments include: Alefacept (AMEVIVE or LFA#TIP, Biogen); Oral retinoids; Cyclosporine; Etanercept and infliximab; topical vitamin D(3) analogues; dithranol

Drugs in clinical investigation are also contemplated, included but not limited to: Amevive (Biogen); BIRB 796 (Boehringer-Ingelheim Pharmaceuticals); Embrel (Amgen); Hectorol (Bone Care International); IDEC-114 (IDEC Pharmaceuticals); LFA-1 inhibitor (Biogen); ONTAK (Ligand Pharmaceuticals); PsorBan (CGC1072)(CellGate); r-IL-18 bp (Serono); and Targretin Gel (Ligand Pharmaceuticals).

(vii) Adjuvants that can be Used in Combination and/or Alternation with the Composition of the Present Invention

In addition, if desired, the composition of the present invention may contain auxiliary substances such as wetting or emulsifying agents, pH buffering agents or adjuvants that enhance the effectiveness of the fraction composition. As non-limiting examples, the plasma or serum fraction can be administered in combination or alternation with any of the following known adjuvants.

Adjumer (PCPP salt; polyphosphazene; polyidi(carboxylatophenoxy)lphosphazene) which may be administered in the soluble form as an adjuvant for parenteral formulations or in the crosslinked form as a microsphere hydrogel for mucosal formulations. It induces a sustained antibody response after a single parenteral immunization and these antibody responses include antigen specific IgG1 and IgG2a. with sustained IgG and IgA responses also induced in after mucosal immunization. Algal Glucan (also known as β-glucan or glucan) is administered with antigen for enhancement of both humoral and cell-mediated immunity. β-Glucans exert their immunostimulatory activities by binding to specific β-glucan receptors on macrophages. This ligand-receptor interaction results in macrophage activation and, in certain formulations, promotes antigen targeting. Algammulin (gamma inulin/alum composite adjuvant) is used in formulations as a primary adjuvant and stimulates immune responses by causing ligation of leukocyte-surface complement receptors (CR) via known biochemical mechanisms, thus placing the antigen close to activated leukocytes. Addition of Algarnmulin is known to enhance both humoral and cell-mediated immunity from either Th1 or Th2 pathways, depending on the weight ratio of inulin to Alhydrogel. Avridine (N,N-dioctadecyl-N′,N′-bis(2-hydroxyethyl) propanediamine; CP20,961) may be incorporated into a liposomal preparation; into aqueous suspensions from alcoholic solution; in Intralipid, an aqueous soybean oil emulsion vehicle; other vegetable and mineral oil vehicles; in Tween 80 dispersions in saline; in saline suspension with alum-precipitated antigen. It has been shown to cause humoral and cellular immunity, proliferation of B and T lymphocytes, protective immunity, activation of macrophages, induction of interferon, enhancement of mucosal immunity when administered orally/enterically with antigen, adjuvanticity with a variety of antigens, induction of IgG2a and IgG2b isotypes. BAY R1005 (N-(2-Deoxy-2-L-leucylamino-β-D-glucopyranosyl)-N-octadecyldodecanoylamide hydroacetate) can be used as a primary adjuvant. BAY R1005 in combination with purified virus vaccines or subunit vaccines led to increased protection of virus-challenged mice. The increase in antibody synthesis induced by BAY R1005 is specifically dependent on the antigen and it acts on the proliferation of B lymphocytes as a second signal which has no effect until the antigen acts as a first signal. BAY R 1005 is capable of activating B lymphocytes without the helper function of T lymphocytes. In mice parenteral immunization with recombinant urease mixed with BAY R1005 induced strong Th1 and Th2 responses and thereby elicited better protection against Helicobacter pylori infection than adjuvants which induced a prominent Th2 type response only (Guy, B., et al., 1998. Systemic immunization with urease protects mice against Helicobacter pylori infection. Vaccine 16:850-856.) Calcitriol (1α, 25-dihydroxyvitamin D3; 1,25-di(OH)₂D3; 1,25-DHCC; 1α, 25-dihydroxycholecalciferol; 9,10-seco(5Z,7E)-5,7,10(19)-cholestatriene-1α,3β,25-triol) has been shown to promote the induction of mucosal and systemic immunity when incorporated into vaccine formulations. Calcium Phosphate Gel has been used as adjuvant in vaccine formulations against diphtheria, tetanus, pertussis and poliomyelitis. It adsorbs soluble antigens and presents them in a particulate form to the immune system and contains no components that are not natural constituents of the body and is very well tolerated. Cholera toxin B subunit (CTB, also known as CTB subunit) augments humoral responses by acting as an efficient carrier/delivery system and is completely non-toxic and has been used extensively in humans without negative side-effects. Cholera holotoxin (CT) has been shown to augment both humoral and cell-mediated immunity, including CTL responses, and thereby enhances MHC class I and II restricted responses. CT exerts immunomodulating effects on T cells, B cells as well as antigen-presenting cells (APC). Cholera toxin A1-subunit-Protein A D-fragment fusion protein (CTA1-DD gene fusion protein) has proven equivalently potent as an adjuvant to the intact cholera holotoxin (CT) for humoral and cell-mediated immunity. CTA1-DD is targeted to B lymphocytes, both memory and naïve cells and acts as a powerful systemic and mucosal adjuvant.

Block Copolymer P1205 (CRL1005) acts as both an adjuvant and stabilizer and forms microparticulate structures that can bind a variety of antigens via a combination of hydrophobic interactions and surface charge. Cytokine-containing Dehydration Rehydration Vesicles (Cytokine-containing Liposomes) induces both cellular and humoral immunity. Dimethyldioctadecylammonium bromide; dimethyldistearylammonium bromide (DDA-CAS Registry Number 3700-67-2) is known for stimulation immune responses against various antigens and especially delayed type hypersensitivity. DHEA (Dehydroepiandrosterone; 5-androsten-3β-ol-17-one; dehydroisoandrosterone; androstenolone; prasterone; transdehydroandrosterone; DHA) can be directly incorporated into vaccine formulations and will enhance antibody formation. DHEA can be administered systemically at the time of vaccination, or can be directly incorporated into the vaccine formulation. DMPC (Dimyristoyl phosphatidylcholine; sn-3-phosphatidyl choline-1,2-dimyristoyl; 1,2-dimyristoyl-sn-3-phosphatidyl choline; (CAS Registry Number 18194-24-6)) and DMPG (Dimyristoyl phosphatidylglycerol; sn-3-phosphatidyl glycerol-1,2-dimyristoyl, sodium salt (CAS Registry Number 67232-80-8); 1,2-dimyristoyl-sn-3-phosphatidyl glycerol) are used in the manufacture of pharmaceutical grade liposomes, typically in combination with DMPG and/or cholesterol and are also used in adjuvant systems for vaccine formulations. DOC/Alum Complex (Deoxycholic Acid Sodium Salt; DOC/Al(OH)₃/mineral carrier complex) is a complex used as adjuvant formulation and is known to enhance the immune response to membrane proteins. Freund's Complete Adjuvant is a mixture of mineral oil (Marco 52) and emulsifier (Arlacel A [mannide monooleate]) as an emulsion of 85% mineral oil and 15% emulsifier with heat-killed antigen. Gamma Inulin is a highly specific activator of the alternative pathway of complement in vitro and in vivo included in adjuvant formulations as a primary adjuvant and also as the immune stimulant when combined as composite particles with alum in the adjuvant Algammulin. It is expected that it stimulates immune responses by causing ligation of leukocyte-surface complement receptors (CR) via known biochemical mechanisms. Addition of gamma inulin is known to enhance both humoral and cell-mediated immunity from both Th1 and Th2 pathways. Gamma inulin also has an antitumor action and an effect on natural immunity. Gerbu Adjuvant, an adjuvant based on GMDP with DDA and Zinc-L-proline have been shown to complex as synergists. GM-CSF (Granulocyte-macrophage colony stimulating factor; Sargramostim (yeast-derived rh-GM-CSF)) is a glycoprotein of 127 amino acids and recombinant human GM-CSF is produced in yeast and it differs from the natural human GM-CSF by substitution of Leu for Arg at position 23. This cytokine is a growth factor that stimulates non-nal myeloid precursors, and activates mature granulocytes and macrophages.

GMDP (N-acetylglucosaminyl-(β1-4)-N-acetylmuramyl-L-alanyl-D-isoglutamine (CAS Registry Number 70280-03-4)-Semi-synthetic. Disaccharide isolated from microbial origin, dipeptide wholly synthetic. U.S. Pat. No. 4,395,399) is known as a primary adjuvant. It has been shown to be an highly effective primary adjuvant in a range of vehicles; aqueous buffers, mineral oil, pluronic/squalane/Tween emulsions. Also effective as oral adjuvant, enhancing mucosal IgA response. Imiquimod (1-(2-methypropyl)-1H-imidazo[4,5-c]quinolin-4-amine; R-837; S26308) can be included in adjuvant formulations as a primary adjuvant component and is known to induce both humoral and cell-mediated immunity via induction of cytokines from monocytes and macrophages. ImmTher™ (N-acetylglucosaminyl-N-acetyhnuramyl-L-Ala-D-isoGlu-L-Ala-glycerol dipalmitate; DTP-GDP) is a potent macrophage activator which induces high levels of TNF, IL-1, and IL-6 both in vitro and in vivo (U.S. Pat. No. 4,950,645). Immunoliposomes prepared from Dehydration-Rehyrdation Vesicles (DRVs) (Immunoliposomes Containing Antibodies to Costimulatory Molecules) are composed of phosphotidylcholine/cholesterol/biotinylate d-phospotidylethanolamine (PC/CH/PEB) in a molar ration of 5:5:1. Antigen is added to the water suspension of DRV followed by repeated vortexing and lyophylization of the liposome suspension. Interferon-γ (Actimmune® (rhIFN-gamma, Genentech, Inc.); immune interferon; IFN-γ; gamma-interferon) has demonstrated higher and earlier neutralizing antibody titers, an increase in duration of neutralizing antibody titers, an increase in MHC class 11 expression on antigen presenting cells, increase in Helper T cell levels, and an improved DTH response. The IFN-gamma is preferably given at the same site and at the same time (within 6 hrs) as the antigen. Interleukin-1β (IL-10; IL-1; human Interleukin 1β mature polypeptide 117-259) is known as a primary adjuvant and is active by oral, intravenous, intraperitoneal and subcutaneous routes. It increases both T-dependent and T-independent responses to different types of antigens and can be active in both primary and secondary responses. Interleukin-2 (IL-2; T-cell growth factor; aldesleukin (des-alanyl-1, serine-125 human interleukin 2); Proleukin®; Teceleukin®) is used as a primary adjuvant, co-emulsified with antigens and lipids, with polyethylene glycol modified long acting form (PEG IL-2), or liposome encapsulated sustained release dosage form. IL-2 supports the growth and proliferation of antigen-activated T lymphocytes and plays a central role in the cascade of cellular events involved in the immune response. Proliferating T-cells also produce a variety of other lymphokines which may modulate other arms of the immune system and in view of these direct and indirect actions of IL-2 on the immune response, IL-2 functions as an adjuvant to vaccination by increasing the specific and durable response to vaccine immunogens. Low doses may give up to 25-fold increase in adjuvant effect, with inhibition of adjuvant effect at high doses. May induce cellular immunity when given systemically, and IgA when administered at a mucosal surface. Interleukin-7 (IL-7) has been shown to enhance antibody production as a primary adjuvant in liposome formulated sustained release form (Bui, et al. “Effect of MTP-PE liposomes and interleukin-7 on induction of antibody and cell-mediated immune responses to a recombinant HIV-envelope protein”, J Acquired Immune Deficiency Syndrome, 1994 August; 7(8):799-806.) and has also been used co-emulsified with antigen and lipids. Interleukin-12 (IL-12; natural killer cell stimulatory factor (NKSF); cytotoxic lymphocyte maturation factor (CLMF)) is used as a primary adjuvant component to enhance Th1-dependent cell-mediated immune responses including cytolytic T-lymphocyte responses.

Immune stimulating complexes (ISCOM(s)™) are a complex composed of typically Quillaja saponins, cholesterol, phospholipid, and antigen in phosphate-buffered saline (PBS). They are antigen-presenting structures that have been shown to generate long-lasting biologically functional antibody response. ISCOMs have demonstrated a protective immunity and a functional cell-mediated immune response, including Class I restricted CTLs have been reported in several systems. They have generally been administered subcutaneously or intramuscularly but non-parenteral administrations (intranasal and oral) have also proven to be effective. Liposomes (L) containing protein or Th-cell and/or B-cell peptides, or microbes with or without co-entrapped interieukin-2, Bis HOP or DOTMA A, [L (Antigen)]; B, [L (IL-2 or DOTMA or Bis HOP+Antigen)]; C, [L (Antigen)-mannose]; D, [L (Th-cell and B-cell epitopes)]; E, [L (microbes)] act as carrier of Th-cell peptide antigen which provides help for co-entrapped B-cell antigen to overcome genetic restriction and induce immunological memory. They may also act as carriers of attenuated or live microbial vaccines to deliver microbes and co-entrapped soluble antigens or cytokines simultaneously to antigen-presenting cells or to protect entrapped vaccines from interaction with maternal antibodies or antibodies to vaccine impurities in preimmunized subjects. Loxoribine (7-allyl-8-oxoguanosine) is known as a primary adjuvant for antibody responses to a wide variety of antigen types in a variety of species. It augments CTL-mediated, NK cell-mediated, macrophage mediated, and LAK cell-mediated cytotoxicity, induces IFN(a/b/γ, TNΦa, TNΦb, IL-1a, IL-6 and up regulates humoral immune responses in immunodeficiency. LT-OA or LT Oral Adjuvant induces both mucosal and systemic immunity (both humoral [including IgA and IgG2, isotypes] and cell-mediated) to killed microorganisms or peptide antigens mixed with it in neutral non-phosphate buffered saline, with/without sodium bicarbonate. MF59 (Squalene/water emulsion—composition: 43 mg/mL squalene, 2.5 mg/mL polyoxyethylene sorbitan monooleate (Polysorbate 80), 2.4 mg/mL sorbitan trioleate (Span 85)) in combination with a variety of subunit antigens results in elevated humoral response, increase T cell proliferation and presence of cytotoxic lymphocytes. MONTANIDE ISA 51 (Purified IFA; Incomplete Freund's adjuvant) addition induces humoral and cell-mediated immunity with various antigens. MONTANIDE ISA 720 (metabolizable oil adjuvant) induces humoral and cell-mediated immunity with various antigens. MPL™ (3-Q-desacyl-4′-monophosphoryl lipid A; 3D-MLA) is used as a primary adjuvant in adjuvant formulations. Its activity is manifested either alone in aqueous solution with antigen, or in combination with particulate vehicles (e.g., oil-in-water emulsions) and its activity may be enhanced by use of vehicle that enforces close association with antigen. MTP-PE (N-acetyl-L-alanyl-D-isoglutaminyl-L-alanine-2-(1,2-dipalmitoyl-sn-glycero-3-(hydroxy-phosphoryloxy)) ethylamide, mono sodium salt) and alternately MTP-PE liposomes are optionally a part of MF59 and are known as immunomodulators. The addition of MTP-PE to the MF59-based HIV vaccine in HIV seropositive individuals resulted in a marked increase in HIV antigen lymphocyte proliferation. Murametide (Nac-Mur-L-Ala-D-Gln-OCH3) induces granulocytosis and enhances the humoral response. Murametide displays the same profile of adjuvant activity as MDP and has been chosen for development because of its favorable therapeutic ratio. When administered in 50% water-in-oil emulsion, it mimics the activity of Freund's complete adjuvant without its side effects (U.S. Pat. No. 4,693,998.) Murapalmitine (Nac-Mur-L-Thr-D-isoGIn-sn-glyceroI dipalmitoyl) is administered in water-in-oil emulsion as an adjuvant of humoral and cell-mediated responses. D-Murapalmitine (Nac-Mur-D-Ala-D-isoGln-sn-glycerol dipalmitoyl) is a strong adjuvant of humoral and cell-mediated immunity when administered in a 50% mineral oil emulsion. NAGO is a mixture of the two enzymes-neuraminidase and galactose oxidase Ag 1:5 ratio in units of activity. It generates cell surface Schiff base-forming aldehydes on antigen presenting cells and Th-cells, thereby amplifying physiologic Schiff base formation that occurs between cell-surface ligands as an essential element in APC:T-cell inductive interaction. It is a potent non-inflammatory adjuvant with viral, bacterial and protozoal subunit vaccines, and is especially effective in the generation of cytotoxic T-cells.

Non-Ionic Surfactant Vesicles (NISV) induces both a humoral and cell-mediated immune response and preferentially stimulates the Th1 sub-population of T-helper cells. It is known to be effective with antigens within a broad size range, from short peptides to particulates, and has extremely low toxicity. Pleuran (β-glucan; glucan) has shown in experimental studies that rabbits as well as mice immunized once by coadministration of viral antigens and 60 μg of Pleuran produced at least 20-fold higher antibody titers than control animals injected with the immunogen alone (Chihara, G. et al., 1989, Lentinan as a host defense potentiator (HDP), Int. J. Immunother. 4:145-154.) PLGA, PGA, and PLA (Homo-and co-polymers of lactic and glycolic acid; Lactide/glycolide polymers; poly-lactic-co-glycolide) used in vaccine delivery have demonstrated an ability to control the release of antigen after administration, thereby eliminating or reducing the need for boost immunizations. Antigens incorporated in PLGA microspheres have exhibited enhanced and prolonged antibody activity responses compared to equivalent doses of free antigen. Pluronic L121 (poloxamer 401) enhances the presentation of antigen to cells of the immune system. PMA (polymethyl methacrylate) is known as a primary adjuvant for all types of antigens. PODDS™ (proteinoid microspheres) serves as a vehicle for oral immunization, protecting the antigen and allowing for co-encapsulation of adjuvants with antigens in microspheres. Poly rA:Poly rU (a double helix comprised of polyadenylic acid and polyuridylic acid) is known as an adjuvant to humoral and cell-mediated immunity when given with antigen; it increases non-specific immunity to microorganisms. Polysorbate 80 may be used in emulsion vaccine formulations including MF59, SAF-1 and Antigen Formulation. Protein cochleates act as both carriers and adjuvants, providing multivalent presentation of antigens to the immune system, with maintenance of native conformation and biological activity and providing protection of antigens from degradation following oral delivery. They stimulate strong mucosal and systemic antibody, proliferative and cytotoxic responses to associated antigens. QS-21 (Stimulon™ QS-21 Adjuvant) can be used in vaccine formulations as a primary adjuvant component for enhancement of both humoral and cell-mediated immunity. Quil-A (Quil-A saponin, Quillaja saponin) is used in veterinary vaccines and for production of ISCOMs. Rehydragel HPA (High Protein Adsorbency Aluminum Hydroxide Gel; alum) and Rehydragel LV (low viscosity alluminum hydroxide gel; alum) are primary adjuvants in parenteral vaccine formulations and aluminum compounds (aluminum hydroxide, aluminum phosphate, alum) are currently the only vaccine adjuvants used in US-licensed vaccines. The use of aluminum adjuvants are accompanied by stimulation of IL-4 and stimulation of the T-helper-2 subsets in mice, with enhanced IgG1 and IgE production. S-28463 (4-Amino-otec,-dimethyl-2-ethoxymethyl-1H-imidazo[4,5-c]quinoline-1-ethanol) induces both humoral and cell-mediated immunity via induction of cytokines from monocytes and macrophages. Experimental results indicate S-28463 is about 100-fold more potent than imiquimod in antiviral models and in cytokine induction from monocytes and macrophages. Syntex Adjuvant Formulation (SAF, SAF-1, SAF-m) causes antigens to arrange on the surface of the emulsion droplets partly because of their amphipathic nature, and partly because of hydrogen bonding with poloxamer 401. The emulsion droplets also activate complement, as demonstrated by consumption of C3 and production of C3b; the latter, on the surface of droplets, targets them to antigen-presenting cells (follicular dendritic cells and interdigitating cells) in lymph nodes of the drainage chain and possibly in more distant lymphoid tissues. In this way the emulsion facilitates the presentation of antigens to responding lymphocytes (threonyl-MDP monograph.) Sclavo peptide (IL-1β 163-171 peptide) enhances immune response to T-dependent and T-independent antigens. It is known as a primary adjuvant and may be administered i.p, i.v., s.c. or p.o and it is active either when administered separately from antigen, or admixed with antigen, or physically linked to antigen. Sendai Proteoliposomes, Sendai-containing Lipid Matrices (Sendai glycoprotein-containing vesicles; fusogenic proteoliposomes; FPLs; Sendai lipid matrix-based vaccines) are potent immunogens and have the ability to stimulate strong T helper and CD8+ cytotoxic T cell responses (CTL) to lipid bilayer-integrated glycoproteins as well as encapsulated peptides, proteins and whole formalin-fixed viruses. These vesicles also act as effective delivery vehicles for drugs and proteins.

Span 85 (Arlacel 85, sorbitan trioleate) is used as an emulsification agent in MF59 adjuvant formulation. Specol (Marcol 52 (mineral oil, paraffins, and cycloparaffins, chain length 13-22 C atoms) Span 85 (emulsifier, sorbitan trioleate) Tween 85 (emulsifier, polyoxyethylene-20-trioleate)) all are individually FDA approved for veterinary use and they function as a depot (slow release of antigen) and a polyclonal activator (independent of presence of antigen) for cells of the immune system (cytokine release). Squalane (Spinacane; Robane®; 2,6,10,15,19,23-hexamethyltetracosane) is a component of Antigen Formulation (AF) and Syntex Adjuvant Formulation (SAF), and constitutes the oil component of the emulsion. Stearyl Tyrosine (octadecyl tyrosine hydrochloride) has adjuvanticity similar to aluminum hydroxide with bacterial vaccines; superior to aluminum hydroxide with viral vaccines. Theramide™ (N-acetylglucosaminyl-N-acetylinuramyl-L-Ala-D-isoGlu-L-Ala-dipalmitoxy propylamide (DTP-DPP)) is a potent macrophage activator and adjuvant. It induces IL-6, IL-12, TNF, IFN-γ, and relatively lessor quantities of IL-10. The compound preferentially induces cellular immunity. Threonyl-MDP (Termurtide™; [thr₁]-MDP; N-acetyl muramyl-L-threonyl-D-isoglutamine) induces the production of a cascade of cytokines, including IL-1a, IL-1β and IL-6. Responding lymphocytes release IL-2 and IFN-γ and the latter increases the production of antibodies of certain isotypes, including IgG2a. This isotype, and the homologous IgG1 in primates, interacts with high affinity Fcγ receptors, so that the antibodies can function efficiently in opsonizing viruses and other infectious agents for uptake by phagocytic cells. Ty Particles or Ty Virus-Like Particles present antigen in a polyvalent, particulate form. Cytotoxic T-lymphocytes are induced in the absence of any other adjuvant formulations. Walter Reed Liposomes (Liposomes containing lipid A adsorbed to aluminum hydroxide, [L(Lipid A+Antigen)+Alum]) have been shown to induce both humoral and cell-mediated immunity. Liposomes containing lipid A provide a very potent adjuvant activity. Adsorption of liposomes containing lipid A to aluminum hydroxide gel contributes additional strong adjuvant activity.

D. Pharmaceutical Compositions

Subjects, such as humans, suffering from disorders characterized by abnormal cell proliferation can be treated by administering to the subject in need thereof an effective amount of the defined plasma or serum fraction in the presence of a pharmaceutically acceptable carrier or diluent. The fraction can be administered by any appropriate route, for example, orally, parenterally, enterally, intravenously, intradermally, subcutaneously, topically, nasally, rectally, in liquid, or solid form.

The plasma or serum fraction is included in the pharmaceutically acceptable carrier or diluent in an amount sufficient to deliver to a patient a therapeutically effective amount to treat cancer without causing serious side effects in the treated patient. Methods to monitor abnormal cell proliferation in vivo and in vitro are well known in the art.

It is to be noted that dosage values will vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The plasma or serum fraction may be administered at once, or may be divided into a number of smaller doses to be administered at varying intervals of time.

A preferred mode of administration of the active compound is through subcutaneous injection, which can optionally include an inert diluent or carrier. Preferred carriers are physiological saline, phosphate buffered saline (PBS) or Ringer's Solution.

If orally administered, the plasma or serum fraction will be lyophilized and will generally include an inert diluent or an edible carrier. It may be enclosed in gelatin capsules or compressed into tablets. For the purpose of oral therapeutic administration, the fraction can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The plasma or serum fraction can also be mixed with other active materials that do not impair the desired action, or with materials that supplement the desired action, such as antibiotics, anti-fungals, anti-inflammatories, protease inhibitors, or other nucleoside or non-nucleoside antiviral agents, as discussed in more detail above. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

If administered by nasal aerosol or inhalation, the plasma or serum fraction is prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

If rectally or vaginally administered in the form of suppositories, the plasma or serum fraction may be prepared by mixing the drug with a suitable non-initiating excipient, such as cocoa butter, synthetic glyceride esters of polyethylene glycols, which are solid at ordinary temperatures, but liquefy and/or dissolve in the rectal cavity to release the drug.

In a preferred embodiment, the plasma or serum fraction is prepared with carriers that will protect it against rapid elimination from the body, such as a controlled release formulation, including implants and micro-encapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. The materials can also be obtained commercially from Alza Corporation.

Liposomal suspensions (including liposomes targeted to infected cells with monoclonal antibodies to viral antigens) are also preferred as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. The plasma or serum fraction is then introduced into the container. The container is then swirled by hand to free lipid material from the sides of the container and to disperse lipid aggregates, thereby forming the liposomal suspension.

### EXAMPLES

Experiments have been conducted to prepare the serum fraction of the present invention and are conducted in vitro to show that the composition of the present invention can inhibit abnormal cell proliferation. Results are summarized below.

### Example 1

### Production of the Inoculant

Patients who are HIV positive and with a detectable viral load, and preferably with a viral load above 2,000, were used to provide blood samples for preparation of an HIV-bearing inoculant. The blood taken from the patient was centrifuged at 32,000 rpm at room temperature using standard sterile laboratory techniques and the resulting patient plasma/serum was frozen at −20° C.

### Example 2

### Inoculation of the Animal

The animal used in the process was first inspected by a veterinarian and evaluated for any underlying abnormalities in the animal and for any pathogens that could cause a possible zoonosis. Once the animal was found to be healthy it was well maintained in a clean environment and monitored by a veterinarian on a regular basis.

The HIV+patient plasma sample prepared as outlined in Example 1 as allowed to thaw to room temperature and approximately 3 cc of the patient plasma/serum was injected subcutaneously into the animal according to standard sterile procedures. Once the animal was injected with the sample, the animal was carefully marked and labeled assigning it a number and indicating where the sample was taken and from whom. A three week period of time was allowed to pass prior to harvesting any of the animal's blood.

### Example 3

### Preparation of the Plasma or Serum Sample

Once the three week period had passed, the specimen animal was injected with 0.5 cc rompun (for ease of handling). After the animal has reached the appropriate level of anesthesia, the external jugular area was sterilely prepped and draped. An 18 gauge one inch needle attached to a 60 cc lure lock syringe was introduced into the external jugular vein and approximately 200 to 400 cc total (using 4-8 lure lock syringes) was sterilely extracted.

The blood was immediately transferred to an ice bath to keep it cool, immediately following which the blood was centrifuged with an office model centrifuge at 32,000×g room temperature and the resultant plasma/serum mixture was sterilely removed and passed through a 0.5 micron suction filtration device. The sterile product as then placed in an ice bath and sterilely filtered through a 0.2 micron suction filtration. It as then transferred to a non-refrigerated ultracentrifuge for twenty minutes at 90,000×g after which the supernatant wassterilely transferred to appropriately sized tubes for a refrigerated ultracentrifuge and spun at 150,000×g for twenty minutes. The supernatant was then passed through an anhydrous filter and then passed through a 0.1 micron suction filtration and into sterile containers where it was then placed into a −70° C. freezer and kept for at least 48 hours. After 48 hours, samples of the batch were taken and cultured both anaerobic ally and aerobically for any pathogens. Once the culture gives negative results, the plasma or serum sample was ready for further processing.

### Example 4

### Antibody Depletion

Antibody depletion experiments were conducted on the product prepared as described in Example 3 by the panning technique as follows. Experiments were completed to remove IgG, IgM and both IgG and IgM. Two wells each of a polystyrene high protein-binding flat-bottomed plate were coated with 100 mL of 1 μg/mL of anti-goat IgG, anti-goat IgM, anti-goat IgG+anti-goat IgM antibodies, or PBS. The plate was incubated for 3 hours at room temperature. Following the incubation the antibodies and PBS were removed and 200 μL of blocking solution (0.05% Tween 20, 10 mg/mL BSA in PBS) was added and the plate was incubated overnight at 4° C. Following the overnight incubation, the wells were washed 5 times with wash buffer (PBS plus 0.05% Tween-20). One hundred fifty microliters (150 μL) of the serum product (designated VR-30) was added to each of the wells and the plate was incubated for 2 hours at 37° C.

The serum is then removed and is used an anti-abnormal cell proliferation assay. In all cases, complete anti-abnormal cell proliferation activity is observed following the treatments, indicating that the removal of goat immunoglobulins (IgG, IgM and IgG+IgM) may not impact the anti-abnormal cell proliferation activity of the test material. Preliminary heat denaturation is conducted and may further suggest that active component(s) is not an antibody. Incubation of VR-30 at 56° C. for 30 min could result in inactivation of specific anti-abnormal cell proliferation activity, while the same treatment of control goat serum might not reduce the level of an observed non-specific inhibition.

### Example 5

### Anti-Abnormal Cell Proliferation Activity of the Initial Plasma or Serum Sample

The anti-abnormal cell proliferation activity of the initial plasma or serum sample is used as a baseline for determining the efficiency of the concentration of the anti-abnormal cell proliferation activity for the fractionation procedures described below.

The anti-abnormal cell proliferation activity of the initial serum sample is determined by: (1) measuring the protein concentration of the serum fraction using Bradford or Lowery based method for determining protein concentration; (2) determining the activity of the fraction in vitro using an anti-abnormal cell proliferation assay (e.g., a breast cancer cell assay or a prostate cancer cell assay); (3) assigning a unit of activity to the fraction based on the amount of protein needed to achieve a 50% inhibition of cell proliferation (IC50). This unit can be used as a baseline to track the fold purification obtained through the fractionation process.

### Example 6

### Anti-HIV Activity of the Initial Plasma or Serum Sample

The anti-HIV activity of the initial plasma or serum sample was used as a baseline for determining the efficiency of the concentration of the anti-HIV activity for the fractionation procedures described below.

The anti-HIV activity of the initial serum sample is determined by: (1) measuring the protein concentration of the serum fraction using Bradford or Lowery based method for determining protein concentration; (2) determining the activity of the fraction using established in vitro anti-viral assay (attachment assay); (3) assigning a unit of activity to the fraction based on the amount of protein needed to achieve a 50% inhibition of cell proliferation (IC50). This unit can be used as a baseline to track the fold purification obtained through the fractionation process.

### Example 7

### Ammonium Sulfate Precipitation

Immunoglobulin G (IgG) is depleted from the sample using a 33% ammonium sulfate precipitation. With the serum sample kept on ice and with constant slow stirring, a saturated ammonium sulfate solution (e.g., 450 g of ammonium sulfate in water to 500 mL) is added to 33% v/v (1 ml saturated ammonium sulfate per 2 mL of serum). The sample is allowed to stir on ice for a period of time ranging from approximately 2 to approximately 4 hours, and then centrifuged for approximately 12,000×g for approximately 20 minutes at 4° C. The supernatant is carefully removed to a clean tube. The precipitate or pellet should contain the majority of the IgG.

The pellet is then washed twice with a volume of ice cold 33% ammonium sulfate solution equivalent to the original volume of the fraction, and then centrifuged at approximately 12,000×g for 20 minutes at 4° C. for each wash. The pellet is then dissolved in a volume of ice cold buffer A equivalent to 10% of the starting volume. The buffer should be suitable for in vitro assays and down stream purification procedures. The suspended pellet is then desalted using a desalting column or dialyzed overnight in ice cold buffer at 4° C. in order to remove any ammonium sulfate.

If a desalting column is used, the procedure involves decanting buffer from the top of column, and loading the sample onto the column. Then, 10 ml of Buffer A is applied to the top of the column and allow to flow through the column. The sample (no more than 3 mL) should be applied to the top of the column and allowed to pass through the column by gravity flow. Fractions of 0.5 ml volume into siliconized tubes.

Then, the 33% ammonium sulfate supernatant produced is fractionated with a 66% ammonium sulfate precipitation. The concentration of the supernatant is adjusted to 66% by adding 1 ml of saturated ammonium sulfate for every 1 ml of supernatant. The 66% precipitation is then performed as above with respect to the 33% precipitation above to provide a 66% pellet. The 66% pellet is then washed twice with a volume of ice cold 66% ammonium sulfate solution equivalent to the original volume of the fraction, and then centrifuged at approximately 12,000×g for 20 min 4° C. for each wash. The pellet is then dissolved in a volume of ice cold buffer A to 10% of the original fraction volume. The suspended pellet is desalted using either a desalting column or by dialysis against buffer A at 4° C. overnight to remove any ammonium sulfate.

Next, the activity of each fraction is tested. The protein concentration of each fraction is measured using the BioRad protein assay. An anti-abnormal cell proliferation assay (e.g., an anti-breast cancer cell or anti-prostate cancer cell) is performed using a concentration of protein equivalent to or less than the amount of protein from the unfractionated material that yielded 50% growth inhibition in the initial assay. A unit of activity is assigned to the fraction based on the amount of protein and the initial dilution needed to achieve a 50% inhibition of growth (IC50). The fold purification is determined by calculating the ratio of activity of the purified material to the starting material. The fold purification is determined by calculating the ratio of the activity of the purified material to the activity of the starting material. This unit should be calculated as activity per mass of total protein and should increase as the purification process is applied.

An analysis of the active fraction or fractions is then performed. Native and denatured PAGE is performed to determine the approximate number and sizes of the proteins in the active fraction(s). An immunoblot analysis is performed to test for the presence of immunoglobulins and albumin in the active fraction.

### Example 8

### DEA Affi-Blue Column Chromatography

DEA Blue Econo-Pac cartridges (BioRad) are used for initial fractionation of the serum sample. These reagents contain an affinity matrix of Cibacron blue dye coupled with a DEAE anion exchanger. The Cibacron blue dye has a high affinity for protein albumins and the DEAE allows for the separation of proteins based on their charge. Immunoglobulins do not bind to the Cibacron blue dye, albumins will bind very tightly, and other proteins should have low to intermediate binding capacity. The various proteins within the serum will also have a range of DEAE binding capacities. The low to intermediate Cibacron blue and the DEAE binding proteins can be eluted from the matrix using competing salt ions. Elution can be performed using a step gradient (as outlined below) or with a linear gradient. The procedure outlined herein details the use of a syringe to load the protein and buffers onto the column; however, the procedure may also be adapted for use with a low or high pressure chromatography system and a larger chromatography column.

(i) Preparation of Buffers

The various buffers needed are prepared. The equilibration and wash buffer (“Buffer A”) contains 28 mM NaCl and 20 mM Tris-HCl pH 8.0. The elution buffers (“Buffer E”) for the DEAE blue cartridge include E1, E2, E5 and E14. E1 is 100 mM NaCl, 20 mM Tris-HCl pH8.0; E2 is 250 mM NaCl 20 mM Tris-HCl pH 8.0; E5 is 500 mM NaCl 20 mM Tris-HCl pH 8.0; E14 is 1.4M NaCl 20 mM Tris-HCl, pH 8.0. Regeneration buffer 1 (Buffer G) is 1.4 M NaCl 100 mM Acetic Acid pH 3.0 40% Isopropanol. Regeneration buffer 2 (“Buffer I”) is 28 mM NaCl 20 mM Tris-HCl 2M Guanidine-HCl. Storage Buffer for DEAE Blue cartridge is 20 mM Sodium Phosphate pH 7.5 0.02% Sodium Azide. Necessary additives for protein stabilization or activity (such as protease inhibitors, glycerol, EDTA, DTT, etc.) may be added to any of the buffers as deemed necessary. The pH listed for the buffers should be the pH at 25° C. All buffers should be chilled to 0-8° C. prior to use to minimize loss of anti-viral activity.

(ii) Sample Preparation

The sample should be in Buffer A. If the starting material is not already in Buffer A, equilibrate the sample with buffer A using a desalting column or by dialysis.

(iii) Preparing the Cartridge for Use

The cartridge is prepared for use by washing it with 10 ml of Buffer G at a flow rate of 1 ml/min to remove any residual dye. It is then washed with 5 ml of buffer E14 at a flow rate of 2 ml/min. A small amount of air may remain just above the upper frit and in the inlet nozzle of the cartridge. The cartridge should be inverted so that the arrow points upward, allowing air to be expelled into the cartridge and out through the outlet nozzle. The cartridge is then washed with Buffer A for 10 minutes at a flow rate of 2.0 ml/min. The cartridge should then be equilibrated with Buffer A for 2 min at 1.0 ml/min. The cartridge should then be inverted, so it points downward.

(iv) Chromatography

The following procedures are used when a syringe is used to load and elute the protein onto the column. (A chromatography system utilizes the same buffers, but they are applied with the system pump and the protein is eluted with a linear salt gradient rather than a step gradient). To load the sample, a sterile syringe is pre-west with Buffer A by sucking and expelling buffer into and out of the syringe. The plunger is removed from the syringe and attached to the cartridge using a luer lock connector. The equilibrated sample is then added to the barrel of the syringe. The plunger is inserted, and the sample is pushed through the cartridge taking care not to inject air into the cartridge. The flow through into a clean siliconized tube is collected as the sample is being loaded. The flow through can be collected into more than one tube.

The cartridge is then washed. The syringe is removed from the cartridge, and washed with buffer A. The plunger is pulled from the syringe and re-attached the barrel to the cartridge. The barrel is then filled with Buffer A and pushed through the cartridge at a flow rate of about 1 ml/min. Wash fractions of 0.5 ml are then collected in siliconized tubes. Wash with a total volume equivalent to 3-5 times the cartridge volume.

The bound proteins are then eluted with a step gradient. The syringe is removed from the cartridge and washed with Buffer E1. The syringe is then attached to the cartridge and pushed through 10 to 15 ml of buffer E1. The fractions are collected in siliconized tubes. The elution steps are then repeated with E-Buffer's of increasing NaCl concentrations. Alternatively, a linear salt gradient from 0 to 500 mM NaCl can be used to elute the bound serum proteins.

The sample is then analyzed. The concentration of protein in each sample is analyzed using the Bio-Rad Protein assay. The peak fractions are analyzed for anti-abnormal cell proliferation activity in vitro, using breast cancer cells and prostate cancer cells. The protein profile of the fractions is analyzed on a polyacrylamide gel. The presence of IgG and IgM are detected by western blot.

### Example 9

### Protein G Affinity Chromatograhy

Protein G is a cell surface protein of group G streptococci. It is a Type III Fc receptor that binds to the Fc region of IgG by a non-immune mechanism. Protein G binds tightly to different subclasses of IgG from a variety of species including human, rabbit, horse, sheep, and goat. IgG immunoglobulins are removed from active fractions using Protein G coupled to a Sepharose matrix. The IgG binds tightly to the matrix and the active non-immunoglobulin fraction will not bind and will be collected in the flow though during application.

An Amersham HiTrap Protein G HP column with a syringe for application of the sample and buffer can be used for Protein G affinity chromatography. The buffers are prepared, including a binding buffer (20 mM Sodium Phosphate, pH 7.0) and an elution buffer (0.1M Glycine-HCl, pH 2.7). Necessary additives for protein stabilization or activity (such as protease inhibitors, glycerol, EDTA, DTT, etc.) may be added to any of the buffers as deemed necessary. The pH listed for the buffers should be the pH at 25° C. All buffers should be chilled to 0-8° C. prior to use to minimize loss of anti-viral activity.

The sample is prepared by adjusting to the composition of Protein G binding buffer using either a desalting column or by dialysis. Use of a desalting column involves decanting buffer from the top of column, and loading the sample onto the column. Then, 10 ml of Buffer A is applied to the top of the column and allow to flow through the column. No more than 3 ml sample should be applied to the top of the column and allowed to pass through the column by gravity flow. Fractions of 0.5 ml volume into siliconized tubes.

If the sample is cloudy or viscous, the buffer adjusted sample is passed through a 0.45 μm filter prior to loading. The column is then prepared. Silicon collected tubes are prepared for the collection of eluted IgG by adding 60-200 l of 1M Tris-HCl pH 9.0 per ml of fraction collected. Tris should not be added to tubes for collection of flow through material. Using a syringe or pump, the column is washed with 10 column volumes of binding buffer.

The sample is then applied to the column and the flow through is collected in siliconized tubes. The flow through will contain the non-IgG proteins. The flow through is collected in 0.5 to 1 mL fractions. The column is washed with 5-10 column volumes of binding buffer. The washed fractions are then collected in siliconized tubes. The bound IgG is then eluted with 2-5 column volumes of elution buffer. The eluted protein material is collected in siliconized tubes containing 1M Tris-HCl, pH 9.0.

The sample is then analyzed. The protein concentration in each sample is quantified using the Bio-Rad Protein assay. The peak fractions are analyzed for anti-abnormal cell proliferation in vitro using breast cancer cells and prostate cancer cells. The protein profile of the fractions is analyzed on a polyacrylamide gel. The fractions are also analyzed for the presence of IgG and IgM by western blot.

### Example 10

### Gel Filtration Chromatography

Gel filtration chromatography is a method of separating molecules based on their size. This procedure may be used at any step in the purification process. Immunoglobulins and serum albumins will fractionate with the higher molecular weight proteins (in the first eluted fractions to come off of the column) and lower molecular weight proteins, such as cytokines, will come off of the column in the latter fractions. Different gel filtration media can be used for separation purposes depending on the size of the protein to be isolated.

The gel filtration media can be Sephacryl S100 or S200 HR. The sample is prepared by adjusting the composition of the gel filtration buffer using either a desalting column or by dialysis. The desalting protocol involves decanting buffer from the top of column, and loading the sample onto the column. Then, 10 ml of Buffer A from Example 7 is applied to the top of the column and allow to flow through the column. No more than 3 ml sample should be applied to the top of the column and allowed to pass through the column by gravity flow. Fractions of 0.5 ml volume into siliconized tubes. If necessary, the sample can be concentrated using a Centricon concentrator (or comparable). If the sample is cloudy or viscous, the buffer-adjusted sample is passed through a 0.45 μm filter prior to loading.

Using a chromatography system, the column is properly attached to the system and equilibrated with gel filtration buffer. chromatography system, properly attach the column to the system and equilibrate the column with GF-buffer (0.05 M Sodium phosphate buffer, pH 7.4, 0.15 M NaCl). The sample is loaded on to the column through the sample loop. Proteins are then eluted with gel filtration buffer at a constant flow rate. The fractions are collected in siliconized tubes.

The samples are analyzed for anti-abnormal cell proliferation activity in vitro against breast cancer cells and prostate cancer cells. The samples are further analyzed for protein profile on a polyacrylamide gel. The samples are also analyzed for the presence of IgG and IgM by western blot.

### Example 11

### Preparation of the Product Using an HIV-Bearing Inoculant

Pathogen-free goats were inoculated with plasma from an uninfected human donor, and plasma from an HIV-infected human donor. Blood was extracted from the goats just prior to inoculation (Week 0) and at weekly intervals up to 5 weeks post inoculation (including a 3 week interval). Serum was prepared from the goat blood.

### Example 12

### Preparation of the Product Using a Breast Cancer Bearing Inoculant and a Prostate Cancer Bearing Inoculant

Pathogen-free goats are inoculated with plasma from an normal human donor, plasma from a human breast cancer patient or plasma from a human prostate cancer patient. Blood is extracted from the goats just prior to inoculation (Week 0) and at weekly intervals up to 5 weeks post inoculation. A serum sample is prepared from the goat blood.

### Example 13

### Partial Fractionation of the Product

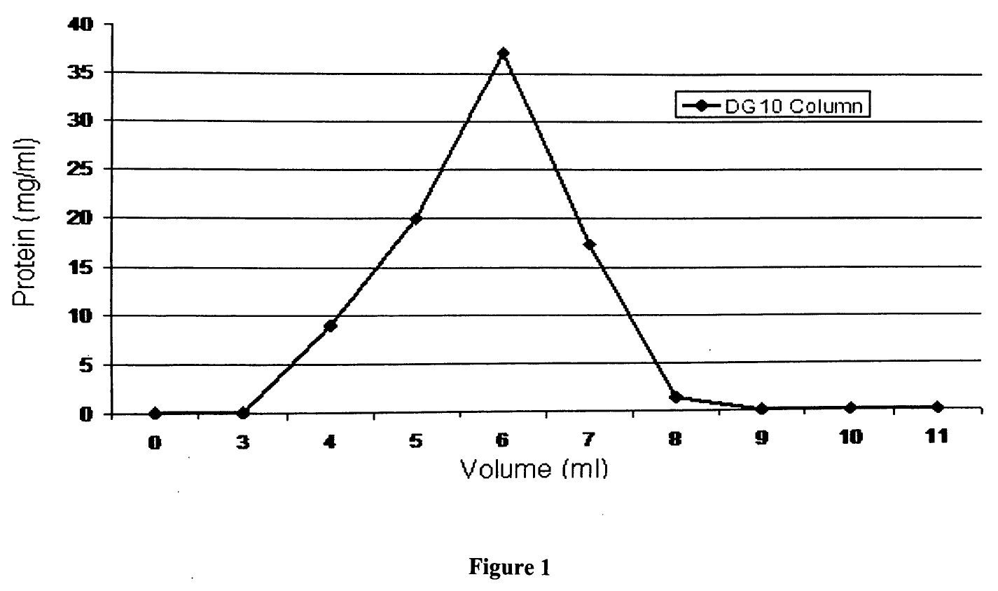

A serum fraction prepared as described in Example 11 was subject to partial fractionation. Specifically, serum was collected at 3 week from a goat inoculated as described in Example 10 (i.e., with 5 ml of plasma from an HIV infected individual). Specifically, serum was collected from animal number 26. The serum was equilibrated with Buffer A (10 mM Tris-HCl pH 8.0, 28 mM NaCl) using a Bio Rad DG-10 gravity flow column at 4° C. Fractions of approximately 1 ml each were collected by hand and analyzed for protein content using the Bio Rad Protein Assay with BSA as a reference standard. The elution profile from the desalting column is shown in FIG. 1.

The fractions for milliliters 4 through 8 from the DG-10 column were pooled and subjected to DEAE-blue chromatography using a Bio Rad 5 ml DEAE-blue cartridge and 20 ml syringe. Sample was loaded onto the cartridge with the syringe and the cartridge was washed with 25 ml of ice cold buffer A (fractions 1 to 35), followed by sequential washing with ice cold buffer A containing 0.1M NaCl (fractions 36 to 50), ice cold buffer A containing 0.5 M NaCl (fractions 51 to 66), and a final elution with ice cold buffer A containing 1.4 M NaCl (fractions 67 to 77). All fractions were collected manually and immediately placed on ice. The protein concentration in each fraction was determined using the Bio Rad Protein assay.

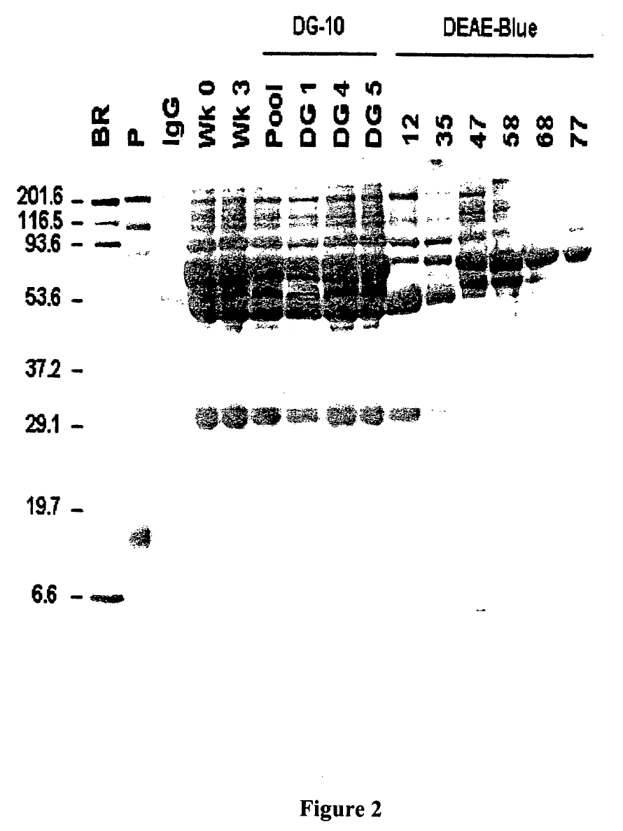

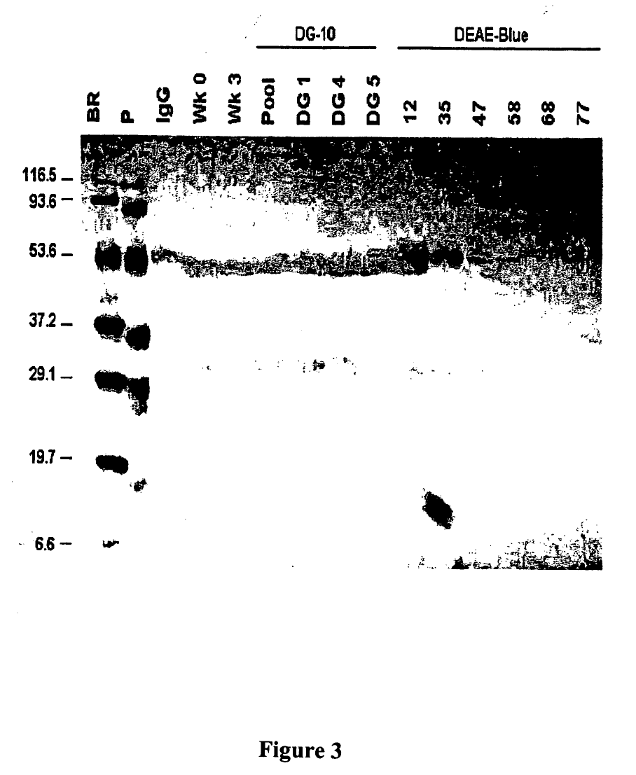

Selected fractions were subjected to SDS-PAGE on 8-16% Bio Rad Criterion gels followed by Coomassie blue staining (FIG. 2) and immunoblot analysis with horse radish peroxidase conjugated rabbit-anti-goat IgG polyclonal antisera (FIG. 3). The following amounts of total protein were loaded onto the gels for both the Coomassie stain and immunoblot gels, 20 μg of total protein for Week 0 serum, Week 3 serum, and DG-10 column fractions (pool, DG-1, DG-4, and DG-5); 10 μg of total protein for DEAE-blue fractions 12, 47, 57, and 68; 5 μg of total protein for DEAE-blue fraction 35; 2.5 μg of total protein for DEAE-blue fraction 77; and 2 μg of total purified total goat IgG (NIH AIDS Research and Reference Reagent Program). Proteins for immunoblot analysis were transferred to 0.45 micron nitrocellulose using a Bio Rad semi-dry transfer apparatus. The membrane was blocked overnight at 4° C. with 5% nonfat milk in PBS-T (PBS plus 0.1% Tween-20) and probed with a 1:2000 dilution of horse radish peroxidase conjugated rabbit-anti-goat IgG polyclonal antibody in PBS-T plus 1% nonfat milk for 1.5 hours at room temperature. Following probing, the membrane was washed extensively with PBS-T and developed with One-step TMB HRP-detection reagent (Pierce) according to the manufacturers instructions.

The serum samples described in Examples 12 is subject to partial fractionation and analysis, as described above for the Example 11 serum sample.

### Example 14

### In Vitro Results

The fractionated compositions prepared above in Example 13 (i.e., the fractionated product of serum samples prepared as described in Examples 11 and 12) are evaluated against breast cancer cells and prostate cancer cells in vitro in order to evaluate the abnormal cell proliferation activity of the goat anti-abnormal cell proliferation product. Results could suggest that the high molecular weight protein-depleted fractions from goats challenged with HIV, breast cancer antigens or prostate cancer antigens possess anti-abnormal cell proliferation activity.

### Example 15

### Partial Fractionation of the Product

The serum fraction prepared as described in Examples 11 was subject to partial fractionation. Specifically, a total of 88 mls of the blood from animal number 26 were removed from the freezer and allowed to thaw on ice.

(i) Ammonium Sulfate Precipitation

The serum sample was divided into 4 polypropylene centrifuge tubes each containing a flea stir bar and placed in an ice bath. A 33% ammonium sulfate precipitation was performed by adding one part of ice cold saturated ammonium sulfate solution per 2 parts serum in 0.5 ml aliquots to each tube with constant stirring. The mixture was allowed to stir on ice for 1.5 hr. Following the 1.5 hrs, the mixture was subjected to centrifugation at 12,000×g for 20 minutes 4° C. The supernatant was transferred to a 400 ml polypropylene beaker and subjected to 66% ammonium sulfate precipitation, as above, with the addition of 1 part saturated ammonium sulfate solution per 1 part supernatant. The pellets from the 33% ammonium sulfate precipitation were washed with 33% ammonium sulfate in PBS and then suspended in 20 mls PBS. The supernatant from the 66% ammonium sulfate precipitation was decanted into 50 ml conical tubes and the pellet was suspended in 20 ml PBS.

The results of the precipitation procedure are shown below in Table I:

TABLE I

| | Volume | Conc. | Total | |
|---|---|---|---|---|
| Fraction | (ml) | (mg/ml) | Protein (mg) | % Protein |
| Initial Bleed Out | 88 | 79.85 | 7026.6 | 100 |
| 33% Wash | 81 | 40.12 | 325 | 4.62 |
| 33% Pellet | 23 | 38.45 | 884.3 | 12.58 |
| 66% Pellet | 39.6 | 88.46 | 3503.1 | 49.85 |
| 66% Sup. | 193.2 | 43.81 | 846.5 | 12.05 |

(ii) DEA-Blue Column Chromatography

Two milliliters of a partially fractionated serum sample previously subjected to 66% ammonium sulfate precipitation and dialysis was further fractionated on a 5 ml BioRad DEAE-Blue column using a BioRad Biologic LP chromatography system.

A DEAE-Blue Econo column (BioRad) was prepared according to the manufacturer's instruction. Briefly, using a syringe, the column was washed with 10 ml of freshly prepared Buffer R (1.4 M NaCl, 0.1M Acetic acid, 40% Isopropanol) followed by washings with 5 ml Buffer B (1.4 M NaCl, 20 mM Tris-HCl pH 7.5, 10% Glycerol) and 30 ml of Buffer A (28 mM NaCl, 20 mM Tris-HCl pH 7.5, 10% Glycerol). The column was then attached to the BioRad BioLogic LP Chromatography system and equilibrated with Buffer A (approximately 50 ml at 1 ml/min). Two milliliters of a dialyzed 66% ammonium sulfate pellet fraction prepared above was thawed on ice and loaded onto the BioLogic LP using a 3 ml syringe and a 2 ml sample loop. The sample was loaded onto the column by running 6 mls of Buffer A at a flow rate of 1 ml/min through the sample loop, and the column was washed with eight column volumes (40 ml) of Buffer A at the same flow rate. The bound proteins were eluted using a linear salt gradient from 28 mM NaCl to 1.4 mM NaCl over 40 mls at 1 ml/min. Eighty two fractions of approximately 0.75 ml each were collected into siliconized 1.5 ml microcentrifuge tubes. Chromatography was monitored using the LP Data View software package. All fractions were stored at −80 C until analyzed.

Samples were analyzed for protein content using the BioRad Protein assay kit with BSA as a quantitative standard. The majority of protein was bound to the column and eluted with the linear gradient; a minority of protein came through in the unbound flow through.

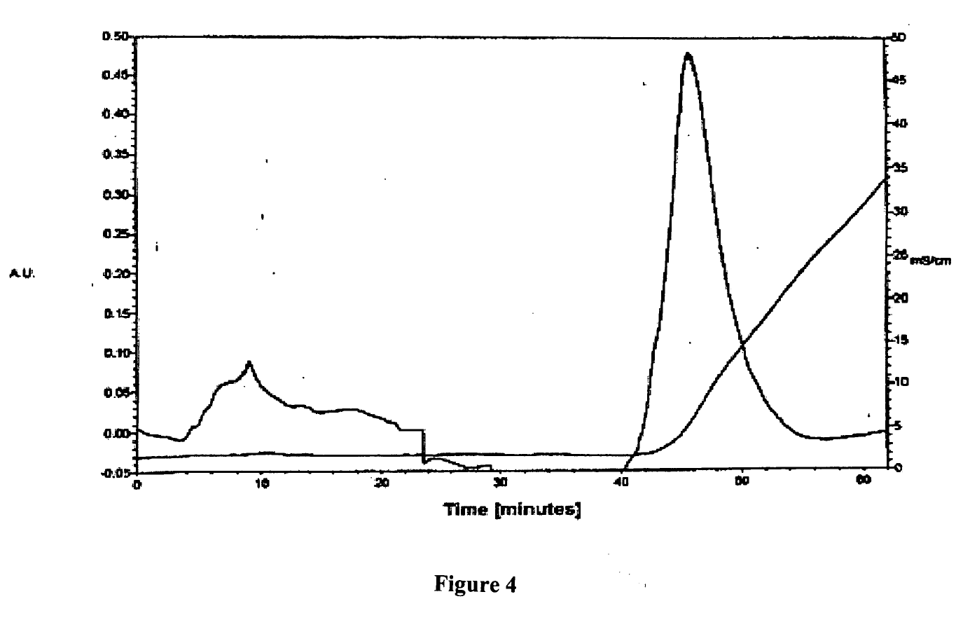

The chromatographic profile of the DEAE-Blue column fractionation of the 66% ammonium sulfate pellet is shown in FIG. 4. Detection of protein occurred four minutes into the run at fraction 6. The concentration of protein in the flow through fractions peaked between 9 and 10 minutes into the run (fraction 13) and then declined until around 26 minutes (fraction 36). The majority of protein in the flow through fractions (concentrations greater than 100 ng/μl) was contained in fractions 9 through 20. A linear salt gradient was initiated 40 minutes into the run and the majority of total protein was eluted between 40 and 54 minutes (fractions 56 to 74). Based on the chromatographic profile, this gradient did not appear to selectively fractionate proteins based on ionic charge.

The serum fraction prepared as described in Examples 12 is subject to fractionation, as described above

(iii) SDS-PAGE

Selected fractions of the serum fraction prepared as described in Example 11 and fractionated as described above were also analyzed by SDS-PAGE. Five micrograms of total protein from selected fractions, including the initial bleed out serum, the dialyzed 66% ammonium sulfate pellet, and DEAE-blue fractions 11-15, and 57-77, were prepared in 2× Lammeli sample buffer, heated to 95° C. for 5 min, and loaded onto a Criterion Precast 8-16% polyacrylamide Tris-HCl gel (Bio Rad). The gel was also loaded with Bio Rad prestained broad range molecular weight markers. The proteins were electrophoresed at 100 V until the bomophenol dye in the sample buffer reached the bottom of the gel. The gel was subjected to Coomassie blue staining with BioSafe Coomassie G250 stain (Bio Rad) according to the manufacturers recommended method. An image of the stained gel was captured on an AlphaImager 2000 and the same software was used to calculate molecular weight of the fractionated proteins.

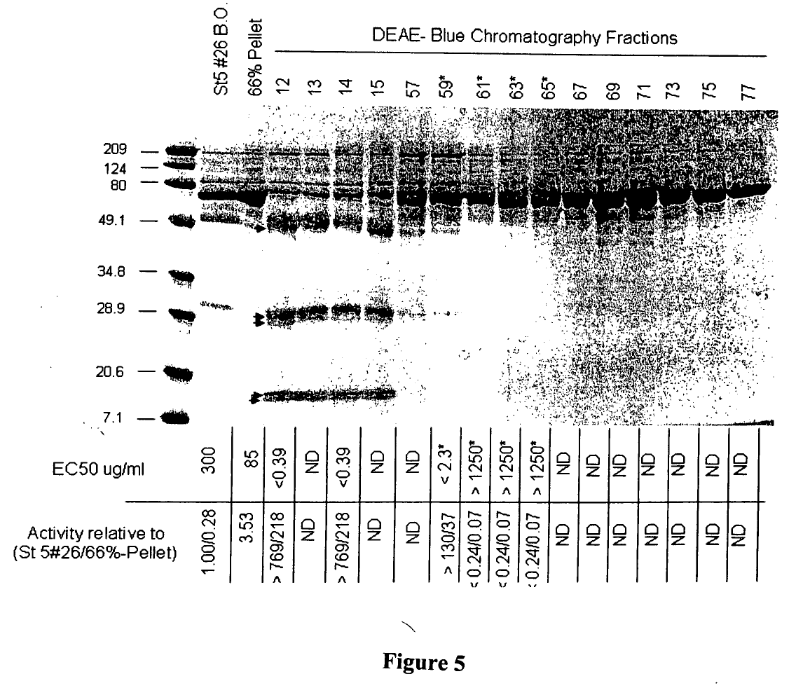

SDS-PAGE analysis and Commassie staining revealed five proteins appear enriched in the unbound fractions. As indicated by the arrow in FIG. 5, five proteins appear enriched in fractions 11 through 15, although other proteins may be present that are not detected by the staining technique. These proteins are (from the top) 49.1, 30.1, 28.6, 14.1, and 12.2 kDa in molecular weight. Light staining bands of equivalent size are found in fractions 57, 59, and 61, but not in fractions 63 through 77.

Selected fractions of the serum fraction prepared as described in Example 12 and fractionated as described above are also analyzed by SDS page. Results could suggest several low molecular weight proteins enriched in the unbound fractions.

### Example 16

### In Vitro Results

A crude serum sample dialyzed against Buffer A, the dialyzed 66% ammonium sulfate pellet, and DEAE-Blue fractions 12, 14, 60, 62, 64, and 66 is analyzed for anti-abnormal cell proliferation activity in an in vitro assay (against breast cancer cells and prostate cancer cells). The results could suggest that the fractions prepared as described in Examples 11 and 12 exhibit anti-abnormal cell proliferation activity.

The invention has been described with reference to its preferred embodiments. Variations and modifications of the invention will be obvious to those skilled in the art from the forgoing detailed description of the invention.

What is claimed is:

1. A plasma or serum fraction for use in the treatment or prevention of a disorder of abnormal cell proliferation or related condition, which fraction is derived from a mammal exposed to an inoculant and which fraction has been depleted of one or more high molecular weight proteins or biological agents present in the unprocessed plasma or serum.

2. The plasma or serum fraction of claim 1, wherein the inoculant is an abnormal cell proliferation-bearing inoculant.

3. The plasma or serum fraction of claim 2, wherein the abnormal cell proliferation-bearing inoculant is a cancer-bearing inoculant.

4. The plasma or serum fraction of claim 3, wherein the cancer-bearing inoculant is the blood, plasma or serum of a patient with cancer.

5. The plasma or serum fraction of claim 3, wherein the cancer-bearing inoculant is a cancer cell or a cancer cell lysate.

6. The plasma or serum fraction of claim 3, wherein the cancer-bearing inoculant is a cancer antigen-bearing inoculant.

7. The plasma or serum fraction of claim 6, wherein the cancer antigen is a breast cancer antigen.

8. The plasma or serum fraction of claim 6, wherein the cancer antigen is a prostate cancer antigen.

9. The plasma or serum fraction of claim 1, wherein the inoculant is an HIV-bearing inoculant.

10. The plasma or serum fraction of claim 1, wherein the inoculant comprises two or more immunogens.

11. The plasma or serum fraction of claim 2, wherein the abnormal cell proliferation-bearing inoculant is selected from the group consisting of atherosclerosis-bearing inoculants, restenosis-bearing inoculants, rheumatoid arthristis-bearing inoculants and psoriasis-bearing inoculants.

12. The plasma or serum fraction of claim 1, wherein the plasma or serum fraction is depleted of from about 50 to about 60% of the immunoglobulin present in the unprocessed plasma or serum.

13. The plasma or serum fraction of claim 1, wherein the plasma or serum fraction is depleted of from about 60 to about 70% of the immunoglobulin present in the unprocessed plasma or serum.

14. The plasma or serum fraction of claim 1, wherein the plasma or serum fraction is depleted of from about 70 to about 80% of the immunoglobulin present in the unprocessed plasma or serum.

15. The plasma or serum fraction of claim 1, wherein the plasma or serum fraction is depleted of from about 80 to about 90% of the immunoglobulin present in the unprocessed plasma or serum.

16. The plasma or serum fraction of claim 1, wherein the plasma or serum fraction has been depleted of from about 90 to about 100% of the immunoglobulin present in the unprocessed plasma or serum.

17. A plasma or serum fraction for use in the treatment or prevention of a disorder of abnormal cell proliferation or related condition, which fraction is derived from a mammal exposed to an inoculant and which fraction has been depleted of two or more high molecular weight proteins present in the whole plasma or serum.

18. The plasma or serum fraction of claim 17, wherein the inoculant is an abnormal cell proliferation-bearing inoculant.

19. The plasma or serum fraction of claim 18, wherein the abnormal cell proliferation-bearing inoculant is a cancer-bearing inoculant.

20. The plasma or serum fraction of claim 19, wherein the cancer-bearing inoculant is the blood, plasma or serum of a patient with cancer.

21. The plasma or serum fraction of claim 19, wherein the cancer-bearing inoculant is a cancer cell or a cancer cell lysate.

22. The plasma or serum fraction of claim 19, wherein the cancer-bearing inoculant is a cancer antigen-bearing inoculant.

23. The plasma or serum fraction of claim 22, wherein the cancer antigen is a breast cancer antigen.

24. The plasma or serum fraction of claim 22, wherein the cancer antigen is a prostate cancer antigen.

25. The plasma or serum fraction of claim 17, wherein the inoculant is an HIV-bearing inoculant.

26. The plasma or serum fraction of claim 17, wherein the inoculant comprises two or more immunogens.

27. The plasma or serum fraction of claim 18, wherein the abnormal cell proliferation-bearing inoculant is selected from the group consisting of atherosclerosis-bearing inoculants, restenosis-bearing inoculants, rheumatoid arthristis-bearing inoculants and psoriasis-bearing inoculants.

28. The plasma or serum fraction of claim 17, wherein the plasma or serum fraction is depleted of immunoglobulin and albumin.

29. The plasma or serum fraction of claim 28, wherein the plasma or serum fraction is depleted of from about 50 to about 60% of the immunoglobulin and albumin present in the unprocessed plasma or serum

30. The plasma or serum fraction of claim 28, wherein the fraction is depleted of from about 60 to about 70% of the immunoglobulin and albumin present in the unprocessed plasma or serum.

31. The plasma or serum fraction of claim 28, wherein the plasma or serum fraction has been depleted of from about 70 to about 80% of the immunoglobulin and albumin present in the unprocessed plasma or serum.

32. The plasma or serum fraction of claim 28, wherein the plasma or serum fraction has been depleted of from about 80 to about 90% of the immunoglobulin and albumin present in the unprocessed plasmas or serum.

33. The plasma or serum fraction of claim 28, wherein the plasma or serum fraction has been depleted of from about 90 to about 100% of the immunoglobulin and albumin present in the unprocessed plasma or serum.

34. A plasma or serum fraction for use in the treatment or prevention of a disorder of abnormal cell proliferation or related condition, which fraction is derived from a mammal exposed to an inoculant and which fraction has been depleted of proteins or biological agents with a molecular weight greater than about 30 kD present in the unprocessed plasma or serum.

35. A plasma or serum fraction for use in the treatment or prevention of a disorder of abnormal cell proliferation or related condition, which fraction is derived from a mammal exposed to an inoculant and which fraction has been depleted of proteins or biological agents with a molecular weight greater than about 50 kD present in the unprocessed plasma or serum.

36. A plasma or serum fraction for use in the treatment or prevention of a disorder of abnormal cell proliferation or related condition, which fraction is derived from a mammal exposed to an inoculant and which fraction has been depleted of proteins or biological agents with a molecular weight greater than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 59, 50, 51, 52, 53, 54, 55, 56, 57, 58, 59, 60, 61, 62, 63, 64 or 65 kD present in the unprocessed plasma or serum.

37. A method for treating or preventing a disorder of abnormal cell proliferation in a subject, comprising administering a therapeutic amount of a plasma or serum fraction derived from a mammal exposed to an inoculant, which fraction has been depleted of one or more high molecular weight proteins present in the unprocessed plasma or serum, either alone or in combination or alternation with another anti-abnormal cell proliferation agent.

38. The method of claim 37, wherein the subject is human.

39. The method of claim 38, wherein the disorder of abnormal cell proliferation is cancer.

40. The method of claim 38, wherein the cancer is selected from the group consisting of bladder, breast, colon, rectal, endometrial, leukemia, lung, melanoma, non-Hodgkin's lymphoma, pancreatic, prostate, skin or thyroid cancer.

41. The method of claim 37, wherein the disorder of abnormal cell proliferation is breast cancer.

42. The method of claim 37, wherein the disorder of abnormal cell proliferation is prostate cancer.

43. The method of claim 37, wherein the disorder of abnormal cell proliferation is a benign tumor.

44. The method of claim 37, wherein the disorder of abnormal cell proliferation is atherosclerosis or restenosis.

45. The method of claim 37, wherein the disorder of abnormal cell proliferation is rheumatoid arthritis.

46. The method of claim 37, wherein the disorder of abnormal cell proliferation is psoriasis.

47. The method of claim 37, wherein the plasma or serum fraction is administered in combination or alternation with an anti-proliferative agent.

48. The method of claim 47, wherein the anti-proliferative agent is a cytotoxic agent.

49. The method of claim 37, wherein the inoculant is selected from the group consisting of cancer-bearing inoculants, atherosclerosis-bearing inoculants, restenosis-bearing inoculants, rheumatoid arthristis-bearing inoculants, psoriasis-bearing inoculants and HIV-bearing inoculants.

50. The method of claim 49, wherein the cancer-bearing inoculant is the blood, plasma or serum of a patient with cancer, a cancer cell or a cancer cell lystate.

51. The method of claim 49, wherein the cancer-bearing inoculant is a cancer antigen-bearing inoculant.

52. The method of claim 51, wherein the cancer antigen is a breast cancer antigen or a prostate cancer antigen.

53. The method of claim 37, wherein the plasma or serum fraction is depleted of immunoglobulin.

54. The method of claim 37, wherein the plasma or serum fraction is depleted of immunoglobulin and albumin.

55. The method of claim 37, wherein the plasma or serum fraction is delivered by subcutaneous administration.

56. The method of claim 37, wherein the plasma or serum fraction is delivered by intravenous or intraarterial administration.

57. A method for treating or preventing a cancer in a subject, comprising administering a therapeutic amount of a plasma or serum fraction derived from a mammal exposed to an inoculant, which fraction has been depleted of one or more high molecular weight proteins or biological agents present in the unprocessed plasma or serum, either alone or in combination or alternation with another anti-cancer agent.

58. The method of claim 57, wherein the inoculant is a cancer-bearing inoculant.

59. The method of claim 57, wherein the cancer-bearing inoculant is a cancer-antigen bearing inoculant.

60. The method of claim 59, wherein the cancer antigen is a breast cancer antigen or prostate cancer antigen.

61. The method of claim 557, wherein the inoculant is an HIV-bearing inoculant.

62. A method of preparing a composition useful in the treatment or prevention of a disorder of abnormal cell proliferation, comprising (a) exposing a mammal to an inoculant; (b) allowing time for the mammal to respond to the inoculant and to produce one or more beneficial biological agents in the blood; (c) obtaining the plasma or serum; (d) processing the plasma or serum to isolate the anti-abnormal cell proliferation activity form one or more high molecular weight protein present in the unprocessed plasma or serum.

63. The method of claim 62, wherein the mammal is an ungulate.

64. The method of claim 63, wherein the mammal is a goat.

65. The method of claim 62, wherein the mammal is not susceptible to infection with the inoculant.

66. The method of claim 62, wherein the inoculant is an abnormal cell proliferation-bearing inoculant selected form the group consisting of cancer-bearing inoculants, atherosclerosis-bearing inoculants, restenosis-bearing inoculants, rheumatoid arthritis-bearing inoculants and psoriasis-bearing inoculants.

67. The method of claim 66, wherein the cancer-bearing inoculant is the blood, plasma or serum of a patient with cancer, a cancer cell or a cancer cell lysate.

68. The method of claim 66, wherein the cancer-bearing inoculant is a cancer antigen-bearing inoculant.

69. The method of claim 68, wherein the cancer antigen is a breast cancer antigen or a prostate cancer antigen.

70. The method of claim 62, wherein the inoculant is an HIV-bearing inoculant.

71. The method of claim 62, wherein the plasma or serum if processed by fractionation.

72. The method of claim 71, wherein the method of fractionation is selected from the group consisting of fractional precipitation, dialysis and ultrafiltration and chromatographic fractionation.

73. The method of claim 71, wherein the fractionation involves a single fractionation step.

74. The method of claim 71, wherein the fractionation involves two or more fractionation steps.

75. The method of claim 74, wherein the fractionation involves two or more different fractionation methods.

76. The method of claim 75, wherein the two or more different fractionation methods include ammonium sulfate precipitation and chromatography.

77. The method of claim 62, wherein the anti-abnormal cell proliferation activity is isolated from immunoglobulin present in the unprocessed plasma or serum.

78. The method of claim 62, wherein the anti-abnormal cell proliferation activity is isolated from the immunoglobulin and albumin present in the unprocessed plasma or serum.

79. The method of claim 62, wherein the anti-abnormal cell proliferation activity is isolated from proteins or biological agents present in the unprocessed plasma or serum with a molecular weight greater than about 50 kD.

80. The method of claim 62, wherein the anti-abnormal cell proliferation activity is isolated from proteins or biological agents present in the unprocessed plasma or serum with a molecular weight greater than about 30 kD.
